(19) United States
(12) Patent Application Publication
(10) Pub. No.: US 2005/0136414 A1
(43) Pub. Date: Jun. 23, 2005

(54) Methods and compositions for making locus-specific arrays

(76) Inventors: Kevin Gunderson, Encinitas, CA (US); David Barker, Del Mar, CA (US); Mark Chee, Del Mar, CA (US); Tim McDaniel, San Diego, CA (US); Robert Yang, San Diego, CA (US)

Correspondence Address: PILLSBURY WINTHROP SHAW PITTMAN LLP, ATTENTION: DOCKETING DEPARTMENT, 11682 EL CAMINO REAL, SUITE 200, SAN DIEGO, CA, 92130, US

(21) Appl. No.: 10/746,249

(22) Filed: Dec. 23, 2003

(51) Int. Cl. C12Q 1/68; C12P 19/34; C12M 1/34

(52) U.S. Cl. 435/6; 435/91.2; 435/287.2

(57) ABSTRACT

The present invention includes methods and compositions relating to locus-specific arrays. More specifically, this invention includes methods for making locus-specific arrays from universal arrays in situ, the custom arrays made using those methods, and methods of using the custom arrays to detect target nucleotides.

### REFERENCE TO GOVERNMENT GRANT

This invention was made with government support under grant CA81952 awarded by the National Institutes of Health. The U.S. government may have certain rights in this invention.

### FIELD OF THE INVENTION

The field of this invention is, generally, arrays for detecting oligonucleotides, and more specifically includes locus-specific arrays made from universal arrays.

### BACKGROUND OF THE INVENTION

Citation of documents herein is not intended as an admission that any of the documents cited herein is pertinent prior art, or an admission that the cited documents are considered material to the patentability of the claims of the present application. All statements as to the date or representations as to the contents of these documents are based on the information available to the applicant and do not constitute any admission as to the correctness of the dates or contents of these documents.

Microarray technology has been applied to a variety of different fields to address fundamental research questions. For example, DNA microarrays can be used to identify polymorphisms, detect mutations, and analyze genetic variations, allowing diagnostic classification and treatment selection. cDNA microarrays are also useful for gene expression analysis, and can be used to correlate the expression of genes or sets of genes with certain physiological processes or medical conditions. Gene expression analysis using microarrays can also aid in medical diagnoses and monitoring the effectiveness of disease therapies.

A universal array is an array of adapter probes having sequences called “addresses,” that are complementary to artificial adapter-specific sequences. The adapter probes on universal arrays can be used to detect adapter sequences that have been attached to target molecules, thereby placing target molecules at known sites on the array, where they are detected and analyzed.

A locus-specific array, on the other hand, is an array of capture probes complementary to target sequences. Creating an array of many different locus-specific capture probes is costly and time-consuming using current methods, due to the relatively high costs of quality control, and to the technology required to generate custom arrays containing specific sequences at different locations. The ability to create locus-specific arrays from universal arrays can result in significant cost savings for manufacturing and quality control, increased flexibility in array design, and decreased experiment-to-experiment variability.

### SUMMARY OF THE INVENTION

The present invention relates to methods for making locus-specific arrays. For example, the invention includes methods for producing locus-specific arrays from universal arrays, locus-specific arrays made from universal arrays, and methods of using the locus-specific arrays to detect and analyze nucleic acids.

The adapter probes of a universal DNA array can be joined to any number of locus-specific nucleotides using the methods of this invention. Thus, this invention includes detecting target analytes having vastly different structural and chemical properties. Using the methods of this invention, a universal array can be converted into a locus-specific array by converting the adapter probes into locus-specific probes in situ, i.e., on the array. The present invention discloses several methods for creating in situ locus-specific arrays from universal arrays. For example, universal arrays can be converted into locus specific arrays in situ using direct immobilization, ligation, polymerase extension, or a combination thereof, to convert adapter probes into locus-specific probes in situ.

Embodiments of the invention include methods for making locus-specific arrays that comprise providing a universal array having a plurality of assay locations. Each assay location can comprise an adapter probe, and at least two of the adapter probes on the universal array can have different sequences. A plurality of chimeric oligonucleotides is provided, where each chimeric oligonucleotide comprises a locus-specific portion and an adapter-specific portion. The chimeric oligonucleotides can be contacted with the adapter probes under conditions for forming a plurality of chimeric-oligonucleotide:adapter-probe hybrids. The resulting hybrids are converted into locus-specific assay locations, thereby converting the universal adapter array into a locus-specific array. Certain embodiments of the invention include methods in which chimeric oligonucleotides are crosslinked to the adapter probe.

The locus-specific portion of the chimeric oligonucleotide can comprise a sequence that is complementary to the locus-specific sequence. This sequence can act as a template in a primer extension or ligation reaction for generating the nascent target capture sequence. The adapter-specific portion of the chimeric oligonucleotide can comprise a sequence that is complementary to the adapter-probe sequence.

Other embodiments include methods in which the adapter probe, having its 5′ end attached to the bead, is extended using a polymerase from its free 3′ terminus. In embodiments, an intervening sequence separates the adapter-specific portion from the locus-specific portion of the chimeric oligonucleotide, and the adapter probe is polymerase-extended using the chimeric oligonucleotide having an intervening sequence as a template. In the embodiments described, the chimeric oligonucleotide can later be denatured from the hybridization complex. Denaturation of the chimeric oligonucleotide results in an extended adapter probe having a single-stranded portion that can serve as a locus-specific probe.

In embodiments of the invention, the chimeric oligonucleotides comprise splint oligonucleotides, each comprising an adapter-specific portion and a locus-specific portion. The splint oligonucleotides can be contacted with the adapter probe and locus-specific oligonucleotides under conditions for forming ternary hybrids, each comprising a adapter probe, splint oligonucleotide and locus-specific oligonucleotide. In embodiments, the adapter probe of the ternary hybrid can be ligated to the locus-specific oligonucleotide to form a ligation-extended locus-specific probe.

In other embodiments, the splint oligonucleotide can contain an intervening sequence between its locus-specific portion and adapter-specific portion. In certain embodiments utilizing a splint oligonucleotide with an intervening sequence, the ternary complex can be immobilized by crosslinking the splint oligonucleotide with the adapter probe and the locus-specific oligonucleotide. In alternate embodiments utilizing a splint oligonucleotide with an intervening sequence, a third oligonucleotide comprising a locus specific portion and a portion complementary to the intervening sequence can be hybridized to the splint oligonucleotide and ligated to the adapter probe. In certain of these embodiments, a polymerase is used to extend from the 3′ end of the adapter probe, across the intervening sequence, to the 5′ end of the locus-specific oligonucleotide, to form an extended adapter probe. Using the methods of the invention, polymerase extension can also be used to extend the adapter probe to the 5′ end of the locus-specific oligonucleotide in the absence of an intervening sequence. In other embodiments utilizing a splint oligonucleotide with an intervening sequence, a fourth oligonucleotide complementary to the sequence between the adapter-probe portion and the locus-specific portion of the splint oligonucleotide can be hybridized to the ternary complex and subsequently ligated at one end to the adapter probe and at the other end to the locus-specific portion of the splint oligonucleotide. In certain of the embodiments employing a splint oligonucleotide, the splint oligonucleotide can be denatured from the hybridization complex. The remaining extended adapter probe can serve as a locus-specific probe.

In other embodiments of the invention, the adapter probe can be hybridized to a splint oligonucleotide, comprising an intervening sequence separating the adapter-specific and locus-specific portions, and a third oligonucleotide hybridized to the locus-specific portion of the splint oligonucleotide. The adapter probe is extended by a nucleic acid polymerase and ligated to the third oligonucleotide. The resulting extended adapter probe can be used to detect a target oligonucleotide. The splint oligonucleotide can be separated from the extended adapter probe before using the extended adapter probe to detect a target oligonucleotide.

The invention also includes methods for converting a universal array to a locus-specific array by hybridizing a chimeric oligonucleotide, comprising an adapter-specific portion and a locus-specific portion, to a locus-specific oligonucleotide in solution prior to contacting the chimeric oligonucleotide with the adapter probe in a universal array. In embodiments, the chimeric oligonucleotide can comprise an intervening sequence separating the locus-specific portion from the adapter-specific portion. In other embodiments it can be a splint oligonucleotide comprising an adapter-specific portion and a locus-specific portion. After the hybrid binds to the array, the locus-specific oligonucleotide can be ligated to the adapter probe nucleotide.

The invention also provides methods for detecting a plurality of loci, comprising providing a universal array having a plurality of assay locations wherein each of the assay locations comprises an adapter probe and the adapter probes are contacted with a plurality of chimeric oligonucleotides, each chimeric oligonucleotide comprising a locus-specific portion and an adapter-specific portion, under conditions for forming a plurality of chimeric oligonucleotide:adapter hybrids. The hybrids are converted into locus-specific assay locations of the locus-specific array, and the locus-specific array is contacted with a plurality of target oligonucleotides under conditions wherein target nucleotides that are complementary to locus-specific assay locations hybridize to the locus-specific assay locations, thereby detecting the target nucleotides.

Embodiments of the invention include a method of making a first and second locus-specific array, comprising the steps of: (a) providing a first universal array having a plurality of assay locations wherein each of the assay locations comprises an adapter probe; (b) providing a first plurality of chimeric oligonucleotides, each chimeric oligonucleotide comprising a locus-specific portion and an adapter-specific portion; (c) contacting the first plurality of chimeric oligonucleotides with the adapter probe under conditions for forming a plurality of chimeric oligonucleotide:adapter probe hybrids; (d) converting the hybrids into locus-specific assay locations of a first locus-specific array; (e) providing a second universal array having a plurality of assay locations comprising the adapter probes; (f) providing a second plurality of chimeric oligonucleotides comprising the adapter-specific portions and locus-specific portions, wherein the locus-specific portions of the first plurality of chimeric oligonucleotides are different from the locus-specific portions in the second plurality of chimeric oligonucleotides; (g) contacting the second plurality of chimeric oligonucleotides with the adapter probes of the second universal array under conditions for forming a second plurality of chimeric oligonucleotide:adapter probe hybrids; and (h) converting the second plurality of hybrids into locus-specific assay locations of a second locus-specific array.

Other embodiments of the invention provide locus-specific arrays, comprising an adapter probe covalently attached to a solid support, a chimeric oligonucleotide comprising an adapter-specific portion and a locus-specific portion, wherein the adapter-specific portion of the chimeric oligonucleotide is hybridized to the adapter probe.

The invention also provides locus-specific arrays comprising an adapter probe covalently attached to a solid support, a locus-specific oligonucleotide, and a splint oligonucleotide comprising an adapter-specific portion and a locus-specific portion wherein the adapter-specific portion of the splint oligonucleotide is hybridized to the adapter probe and the locus-specific portion of the splint oligonucleotide is hybridized to the locus-specific oligonucleotide.

Additionally, the invention provides locus-specific arrays comprising an adapter probe covalently attached to a solid support, and a chimeric oligonucleotide comprising an adapter-specific portion and a locus-specific-portion, wherein the adapter-specific portion of the chimeric oligonucleotide is hybridized to the adapter probe. Embodiments of the invention provide locus-specific arrays comprising an adapter probe covalently attached to a solid support and a chimeric oligonucleotide comprising an adapter-specific portion and a locus-specific portion, wherein the adapter-specific portion of the chimeric oligonucleotide is crosslinked to the adapter probe.

The invention further includes kits comprising universal arrays having a plurality of assay locations wherein said assay locations comprise an adapter probe and a plurality of chimeric oligonucleotides, wherein each chimeric oligonucleotide comprises a locus-specific oligonucleotide and an adapter-specific portion having a sequence complementary to at least one of the adapter probes, and methods for using the kits.

### BRIEF DESCRIPTION OF THE DRAWINGS

FIG. 1 Schematic diagram illustrating an embodiment of the invention in which an adapter probe on a universal array is converted into a locus-specific probe by hybridization extension. The adapter probe, attached to a solid substrate, is hybridized to a chimeric oligonucleotide having an adapter-specific sequence and a locus-specific sequence. The locus-specific sequence can bind to a target sequence of a target nucleic acid.

FIG. 2 Schematic diagram illustrating an embodiment of the invention in which an adapter probe on a universal array is converted into a locus-specific probe by polymerase extension of the adapter probe. A chimeric oligonucleotide having an adapter-specific sequence and a locus-specific sequence is hybridized to the adapter probe by its adapter-specific sequence. The adapter probe is extended to add a locus-specific sequence by a polymerase, using the locus-specific sequence of the chimeric oligonucleotide as template. The polymerase-extended portion of the adapter probe is complementary to a target sequence of a target nucleic acid.

FIG. 3 Schematic diagram illustrating an embodiment of the invention in which an adapter probe on a universal array is converted into a locus-specific probe by ligation of a third oligonucleotide to the adapter probe. A splint oligonucleotide having an adapter-specific sequence and a locus-specific sequence is hybridized to the adapter probe by its adapter-specific sequence. The third oligonucleotide, having the locus-specific sequence complementary to the locus-specific sequence of the splint oligonucleotide, is hybridized to the splint oligonucleotide, then ligated to the adapter probe. A ligation-extended locus-specific adapter probe results. In embodiments, the locus-specific sequence of the third oligonucleotide can be longer than the locus-specific portion of the splint oligonucleotide.

FIG. 4 Schematic diagrams illustrating certain embodiments of the invention in which adapter probes on universal arrays are converted into locus-specific probes using chimeric or splint oligonucleotides having an intervening sequence. A. Ligation of adapter probe to a third oligonucleotide having a locus-specific sequence and a sequence complementary to the intervening sequence. B. Ligation of adapter probe to a fourth oligonucleotide, having a sequence complementary to the intervening sequence. The fourth oligonucleotide is ligated to the third oligonucleotide, having a locus-specific sequence. In embodiments, the locus-specific sequence of the third oligonucleotide can be longer than the locus-specific portion of the splint oligonucleotide. C. Crosslinking of a chimeric or splint oligonucleotide to the adapter probe and a third oligo having a locus-specific sequence. Alternatively, a splint oligonucleotide can have a second adapter-specific sequence. D. Polymerase extension of the adapter probe. The resulting extended adapter probe contains the sequence complementary to the intervening sequence and a portion complementary to a target nucleic acid. E. Polymerase extension of the adapter probe and ligation of the extended adapter probe to a third oligonucleotide. This embodiment can be used with a splint oligo not containing an intervening sequence.

FIG. 5 Schematic diagram illustrating an embodiment of the invention in which an adapter probe on a universal array is converted into a locus-specific probe by ligation of a splint oligonucleotide:third oligonucleotide hybrid to the adapter probe. The chimeric oligonucleotide is hybridized to the third, locus-specific oligonucleotide prior to contacting the adapter probe on the universal array. In embodiments, the locus-specific sequence of the third oligonucleotide can be longer than the locus-specific portion of the splint oligonucleotide.

FIG. 6 Polymerase extension efficiency data obtained using Sets A and B target oligonucleotides.

FIG. 7 Polymerase extension efficiency data obtained using Set C target oligonucleotides.

### DETAILED DESCRIPTION OF THE INVENTION

In describing the present invention, the following terms will be employed, and are intended to be defined as indicated below. Unless otherwise indicated, all terms used herein have the same ordinary meaning as they would to one skilled in the art of the present invention.

Definitions

As used herein, the term “array” is intended to mean a group of elements forming a unit. When used in reference to particles, the term is intended to mean a group of particles that can be independently separable but combine as basic elements to form a larger aggregate. An array can include, for example, a two-dimensional or three-dimensional arrangement of particle elements as well as higher order multi-dimensional arrangements of particle elements. The term “random” when used in reference to an array is intended to mean that the arrangement of particles within an aggregate lacks a predetermined organization. The term “order” or “ordered” when used in reference to a random array is intended to mean that the organizational arrangement of particles within a random array has been determined. Therefore, a random array can become ordered once the location or position of a particle is known.

Exemplary microarrays that can be used in the invention include, without limitation, those described in Butte, Nature Reviews Drug Discov. 1:951-60 (2002) or U.S. Pat. Nos. 6,287,768; 6,288,220; 6,287,776; 6,297,006 and 6,291,193, all hereby expressly incorporated by reference. Further examples of array formats that are useful in the invention are described in U.S. Pat. No. 6,355,431 B1, U.S. 2002/0102578 and PCT Publication No. WO 00/63437, all hereby expressly incorporated by reference. Exemplary formats that can be used in the invention to distinguish beads in a fluid sample using microfluidic devices are described, for example, in U.S. Pat. No. 6,524,793, hereby expressly incorporated by reference.

Arrays useful in practicing the present invention are known and used in the art and have been described in numerous publications. A high-density array can be an array of arrays or a composite array having a plurality of individual arrays that is configured to allow processing of multiple samples. Such arrays allow multiplex detection. Exemplary composite arrays that can be used in the invention, for example, in multiplex detection formats are described in U.S. Pat. No. 6,429,027, and U.S. 2002/0102578, hereby expressly incorporated by reference. Each individual array can be present within each well of a microtiter plate. Thus, depending on the size of the microtiter plate and the size of the individual array, very high numbers of assays can be run simultaneously; for example, using 96 individual arrays each having 2,000 assay locations such as in a 96 well microtiter plate format, 192,000 assays can be performed in parallel; the same number of assay locations used in a 384 microtiter plate format yields 768,000 simultaneous assays, and a format utilizing a 1536 microtiter plate gives 3,072,000 assays.

An “assay location” as used herein refers to an identifiable position of an array that can interact with an analyte such that the analyte can be detected. Exemplary assay locations include, without limitation, populations of probes attached to form features on a printed array, particles attached to a solid surface or aligned in a fluid stream or other formats exemplified herein or known in the art.

The term “universal array” describes an array of adapter probes that are complementary to artificial target adapter sequences. The adapter probes are capable of being joined to ligands that bind to any number of target analytes including, e.g., nucleic acids, oligonucleotides, peptides, and small molecules. Thus, the same array can be used for vastly different target analytes. A universal array can include adapter probes having sequences that are designed to lack complements to sequences found in a particular population of target oligonucleotides. In particular embodiments, a universal array can lack complements to sequences found in a genome of a particular organism including, but not limited to, a mammal such as a rodent, mouse, rat, rabbit, guinea pig, ungulate, horse, sheep, pig, goat, cow, cat, dog, primate, human or non-human primate; a plant such as Arabidopsis thaliana, corn (Zea mays), sorghum, oat (oryza sativa), wheat, rice, canola, or soybean; an algae such as Chlamydomonas reinhardtii; a nematode such as Caenorhabditis elegans; an insect such as Drosophila melanogaster, mosquito, fruit fly, honey bee or spider; a fish such as zebrafish (Danio rerio); a reptile; an amphibian such as a frog or Xenopus laevis; a dictyostelium discoideum; a fungi such as pneumocystis carinii, Takifugu rubripes, yeast, Saccharamoyces cerevisiae or Schizosaccharomyces pombe; or a plasmodium falciparum. A universal array can also include probes that lack complements to sequences found in smaller genomes such as those from a prokaryote such as a bacterium, Escherichia coli, staphylococci or mycoplasma pneumoniae; an archae; a virus such as Hepatitis C virus or human immunodeficiency virus; or a viroid. If desired, a universal array can lack complements to sequences expressed by a particular organism such as one or more of the organisms set forth above.

The term “locus-specific array” describes an array of capture probes that are complementary to sequences found in a particular population of target oligonucleotides. In particular embodiments, a locus-specific array can have complements to sequences found in a genome of a particular organism including, but not limited to, a mammal such as a rodent, mouse, rat, rabbit, guinea pig, ungulate, horse, sheep, pig, goat, cow, cat, dog, primate, human or non-human primate; a plant such as Arabidopsis thaliana, corn (Zea mays), sorghum, oat (oryza sativa), wheat, rice, canola, or soybean; an algae such as Chlamydomonas reinhardtii; a nematode such as Caenorhabditis elegans; an insect such as Drosophila melanogaster, mosquito, fruit fly, honey bee or spider; a fish such as zebrafish (Danio rerio); a reptile; an amphibian such as a frog or Xenopus laevis; a dictyostelium discoideum; a fungi such as pneumocystis carinii, Takifugu rubripes, yeast, Saccharamoyces cerevisiae or Schizosaccharomyces pombe; or a plasmodium falciparum. A locus-specific array can also include probes that complement sequences found in smaller genomes such as those from a prokaryote such as a bacterium, Escherichia coli, staphylococci or mycoplasma pneumoniae; an archae; a virus such as Hepatitis C virus or human immunodeficiency virus; or a viroid. If desired, a locus-specific array can include complements to sequences expressed by a particular organism such as one or more of those set forth above. A locus-specific array can include oligonucleotide probes that are complementary to sequences in one or more nucleic acids expressed at a particular developmental stage, at a particular metabolic stage, in a pathological condition, or in response to a particular environment or stimulus.

“Nucleic acid,” “oligonucleotide” or grammatical equivalents as used herein refer to at least two nucleotides covalently linked together. A nucleic acid of the present invention will generally contain phosphodiester bonds, although in some cases, as outlined below, nucleic acid analogs are included that may have alternate backbones, comprising, for example, phosphoramide (Beaucage et al., Tetrahedron 49(10):1925, 1993) and references therein; Letsinger, J. Org. Chem. 35:3800, 1970; Sprinzl et al., Eur. J. Biochem. 81:579, 1977; Letsinger et al., Nucl. Acids Res. 14:3487, 1986; Sawai et al., Chem. Lett. 805, 1984, Letsinger et al., J. Am. Chem. Soc. 110:4470, 1988; and Pauwels et al., Chemica Scripta 26:141, 1986, all hereby expressly incorporated by reference); phosphorothioate (Mag et al., Nucleic Acids Res. 19:1437, 1991; and U.S. Pat. No. 5,644,048, hereby expressly incorporated by reference), phosphorodithioate (Briu et al., J. Am. Chem. Soc. 11 1:2321, 1989, hereby expressly incorporated by reference); O-methylphophoroamidite linkages (see Eckstein, Oligonucleotides and Analogues: A Practical Approach, Oxford University Press, hereby expressly incorporated by reference), and peptide nucleic acid backbones and linkages (see Egholm, J. Am. Chem. Soc. 114:1895, 1992; Meier et al., Chem. Int. Ed. Engl. 31:1008, 1992; Nielsen, Nature, 365:566, 1993; Carlsson et al., Nature 380:207, 1996, all of which are incorporated by reference). Other analog nucleic acids include those with positive backbones (Denpcy et al., Proc. Natl. Acad. Sci. USA 92:6097, 1995, hereby expressly incorporated by reference); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423, 1991; Letsinger et al., J. Am. Chem. Soc. 110:4470, 1988; Letsinger et al., Nucleoside & Nucleotide 13:1597, 1994; Chapters 2 and 3, ASC Symposium Series 580, “Carbohydrate Modifications in Antisense Research”, Eds. Y. S. Sanghui and P. Dan Cook; Mesmaeker et al., Bioorganic & Medicinal Chem. Left. 4:395, 1994; Jeffs et al., J. Biomolecular NMR 34:17, 1994; Tetrahedron Left. 37:743, 1996, all of which are hereby expressly incorporated by reference); and non-ribose backbones, including those described in U.S. Pat. Nos. 5,235,033 and 5,034,506, and Chapters 6 and 7, ASC Symposium Series 580, “Carbohydrate Modifications in Antisense Research,” Eds. Y. S. Sanghui and P. Dan Cook, all hereby expressly incorporated by reference. Nucleic acids containing one or more carbocyclic sugars are also included within the definition of nucleic acids. See Jenkins et al., Chem. Soc. Rev. 169-176, 1995, hereby expressly incorporated by reference). Several nucleic acid analogs are described in Rawls, C & E News Jun. 2, 1997 page 35. All of these references, including the analogs described therein are hereby expressly incorporated by reference. These modifications of the ribose-phosphate backbone may be made to facilitate the addition of labels to the oligonucleotides of the invention, or to increase the stability and half-life of such molecules in physiological environments.

The term “complementary” is intended to describe absolute base pair matching as well as homologous base pair matching allowing hybridization under selected hybridization conditions such as stringent hybridization conditions known to those of skill in the art including, for example, those set forth in further detail below.

“Adapter probes” are oligonucleotides that will specifically hybridize under selected conditions to all or part of a complementary adapter-specific sequence. In one embodiment, the universal array comprises at least two different adapter probes, each at a different assay location.

In other embodiments, the adapter probes are at least about 8, 10, 12, 15, 18, 20, 22, 25, 30, 35, 40, 45, 50, 55, 60, 65, 70, 75 or more base pairs in length.

An “adapter-specific portion,” when used in reference to an oligonucleotide, means a sequence of the oligonucleotide that is identical or complementary to an adapter probe. Complementarity or identity as used in the terms can be perfect or less than perfect such as at least about 99%, 97%, 95% or 90% complementary to or identical with a second sequence.

An adapter-specific probe or portion typically has sufficient complementarity to at least part of an adapter-probe sequence to hybridize under selected conditions to at least part of the adapter probe, including, for example, stringent conditions known to those of skill in the art or exemplified below.

A “locus” is a sequence in a nucleic acid having a known location in the nucleic acid. A nucleic acid having a locus can be a genomic DNA or RNA, mRNA, tRNA, rRNA or other nucleic acid found in or isolatable from a biological organism.

A “locus-specific portion” refers to a sequence of an oligonucleotide that is identical or complementary to a locus in a nucleic acid sequence. Thus, a locus-specific oligonucleotide or portion thereof has sufficient complementarity to at least part of a target locus sequence to hybridize to it under selected conditions. A locus-specific oligonucleotide or portion thereof can have perfect complementarity to a locus sequence or lesser degrees of complementarity such as at least about 99%, 97%, 95% or 90% complementarity so long as a hybrid can form under selected conditions.

The term “hybrid” refers to two nucleic acids or nucleic acid portions that are associated with each other via hydrogen bonds between complementary base pairs.

When the phrase “hybridizing the adapter-specific portion of the splint oligonucleotide to the adapter probes” is used, it is meant that the adapter-probe oligonucleotide sequence, to which the adapter-specific portion of the chimeric oligonucleotide is complementary, is fully or partially hybridized. When the phrase “hybridizing the locus-specific portion of the splint oligonucleotide to the locus-specific oligonucleotide” is used, it is meant that the locus-specific oligonucleotide sequence, to which the locus-specific portion or region of the splint oligonucleotide is complementary, is fully or partially hybridized. Either part or all of the probe sequence may be hybridized in various embodiments contemplated by the invention.

A “chimeric oligonucleotide” is an oligonucleotide having an adapter-specific portion and a second portion that is specific for a different sequence. The second portion of the chimeric oligonucleotide can be, for example, a locus-specific portion or second adapter-specific portion. A “splint oligonucleotide” refers to a chimeric oligonucleotide having a first adapter-specific portion and a second portion which can hybridize to a third oligonucleotide containing a locus specific portion. An “adapter splint oligonucleotide” refers to a chimeric oligonucleotide having a first adapter-specific portion and a second adapter-specific portion. An adapter splint oligonucleotide can bind to an adapter probe of an array by hybridization of the first adapter-specific portion with the adapter probe and can also bind to a chimeric oligonucleotide by hybridization of the second adapter-specific sequence with an adapter-specific sequence of the chimeric oligonucleotide. Accordingly, a ternary complex can be formed between an adapter probe, splint oligonucleotide and chimeric oligonucleotide, thereby forming a sandwich structure. Such a ternary complex can be further modified using methods exemplified herein for adapter probe:chimeric oligonucleotide duplex hybrids, including, for example, polymerase extension, ligation, or both. A “locus splint oligonucleotide” refers to a chimeric oligonucleotide having a first adapter-specific portion and a second locus specific portion. A locus splint oligonucleotide can bind to an adapter probe of an array by hybridization of the first adapter-specific portion with the adapter probe and can also bind to a chimeric oligonucleotide or a third oligonucleotide containing a locus specific portion by hybridization of the locus specific sequence of the locus splint oligonucleotide with a locus specific sequence on a third oligonucleotide or chimeric oligonucleotide.

In some embodiments, the chimeric oligonucleotide, or the splint oligonucleotide, has an intervening sequence between the adapter-specific portion and the locus-specific portion, or between the first adapter specific portion and the second adapter specific portion.

A “solid support” as used herein refers to any material that can be modified to contain discrete individual sites appropriate for the attachment or association of beads and is amenable to at least one detection method. As will be appreciated by those in the art, the number of possible substrates is very large. Possible substrates include, but are not limited to, glass and modified or functionalized glass, plastics (including acrylics, polystyrene, polyacrylamide, and copolymers of styrene and other materials, polypropylene, polyethylene, polybutylene, polyurethanes, TeflonJ, etc.), polysaccharides, nylon or nitrocellulose, resins, silica or silica-based materials including silicon and modified silicon, carbon, metals, inorganic glasses, plastics, optical fiber bundles, and a variety of other polymers. In general, the substrates allow optical detection and do not themselves appreciably fluoresce.

The term “target oligonucleotide,” “target nucleic acid” or grammatical equivalents herein refer to a nucleic acid having a target sequence on a single strand of nucleic acid that is detected or for which detection is desired. Examples of target oligonucleotides include, but are not limited to, genome fragments, mRNA molecules, cDNA molecules, chromosomes, and rRNA molecules. Further examples of target sequences are gene sequences, mRNA sequences, regulatory sequences, or typable loci such as single nucleotide polymorphisms (SNPs), mutations, variable number of tandem repeats (VNTRs) and single tandem repeats (STRs), other polymorphisms, insertions, deletions, splice variants or any other known genetic markers. Exemplary resources that provide known SNPs and other genetic variations include, but are not limited to, the dbSNP administered by the NCBI and available online at ncbi.nlm.nih.gov/SNP/ and the HCVBASE database described in Fredman et al. Nucleic Acids Research, 30:387-91, (2002) and available online at hgvbase.cgb.ki.se/., hereby expressly incorporated by reference.

Modes of Carrying out the Invention

According to the methods of the invention, locus-specific arrays can be made using universal arrays having assay locations at which a known adapter probe or probes are attached. These universal array locations can be converted through oligonucleotide hybridization, polymerase extension, and/or ligation, thereby converting them into locus-specific array locations. The arrays are useful for detecting the presence of a particular locus, for example, in a genotyping method. The arrays are also useful for determining the amount of a particular sequence in a test sample, for example, in a gene expression analysis method.

An advantage of the methods of the invention is that they can be used to increase the throughput and efficiency of making locus-specific arrays. The design and synthesis of locus-specific oligonucleotide arrays is relatively time-consuming and costly using current methods in which, for example, new oligonucleotides are synthesized at discreet locations of an array. By comparison, soluble populations of the same oligonucleotides can typically be designed and synthesized rapidly and at reduced cost. In accordance with the present invention, a plurality of different locus-specific or custom arrays can be synthesized by designing a single universal array, repeatedly synthesizing new batches of the same array, and subsequently converting the universal arrays with different populations of soluble oligonucleotides. Thus, the time, resources and costs associated with directly synthesizing new populations of oligonucleotides at discreet locations of arrays can be reduced by making a single universal array, and introducing variability in the form of custom synthesized populations of soluble oligonucleotides.

In certain embodiments of the invention, the universal array locations can be converted into locus-specific assay locations of a locus-specific array through the hybridization of chimeric oligonucleotides to the adapter probes of a universal array. Generally, an array of arrays can be configured in any of several ways. For example, a one-component system can be used. That is, a first substrate having a plurality of assay locations, such as a microtiter plate, can be configured such that each assay location contains an individual array. Thus, the assay location and the array location can be the same. For example, the plastic material of a microtiter plate can be formed to contain a plurality of bead wells in the bottom of each of the assay wells. Beads containing the adapter probes of the invention can then be loaded into the bead wells in each assay location.

Alternatively, a two-component system can be used. In a two-component system, individual arrays can be formed on a second substrate, which then can be fitted or dipped into locations on a first substrate such as a first microtiter plate substrate to form a universal array. For example, fiber optic bundles can be used as individual arrays, generally with bead wells etched into one surface of each individual fiber, such that the beads containing the adapter probes are loaded onto the end of the fiber optic bundle. The composite array thus includes a number of individual arrays that are configured to fit within the wells of a microtiter plate.

Accordingly, a universal array from which a locus-specific array is made using the methods of the invention can be a composite array having a substrate with a surface having multiple assay locations. Any of a variety of arrays having a plurality of candidate agents in an array format can be used as the universal array in the invention. The size of an array used as the universal array in the present invention can vary depending on the probe composition and desired use of the array. Arrays containing from 2 different probes to many millions can be made, with very large fiber optic arrays being possible. Generally, an array can have from two to as many as a billion or more array locations per square cm. An array location can be, for example, an area on a surface to which a probe or population of similar probes are attached or a particle. In the case of a particle, its array location can be a fixed coordinate on a substrate to which it is attached or a relative coordinate compared to locations of one or more other reference particles in a fluid sample such as a stream passing through a flow cytometer. Very high density arrays can serve as universal arrays in the invention including, for example, those having from about 10,000,000 array locations/cm² to about 2,000,000,000 array locations/cm² or from about 100,000,000 array locations/cm² to about 1,000,000,000 array locations/cm². High density arrays can also be used including, for example, those in the range from about 100,000 array locations/cm² to about 10,000,000 array locations/cm² or about 1,000,000 array locations/cm² to about 5,000,000 array locations/cm². Moderate density arrays useful in the invention can range, e.g., from about 10,000 array locations/cm² to about 100,000 array locations/cm², or from about 20,000 array locations/cm² to about 50,000 array locations/cm². Low density arrays are generally less than 10,000 particles/cm² with from about 1,000 array locations/cm² to about 5,000 array locations/cm² being useful, for example. Very low density arrays having less than 1,000 array locations/cm², from about 10 array locations/cm² to about 1000 array locations/cm², or from about 100 array locations/cm² to about 500 array locations/cm² are also useful in some applications. If desired, arrays having multiple substrates can be used, including, for example substrates having different or identical compositions. Thus for example, large arrays can include a plurality of smaller substrates.

The number of locus-specific arrays made using the methods of the invention can be set by the size of the microtiter plate used; thus, 96 well, 384 well and 1536 well microtiter plates can be used with composite arrays comprising 96, 384 and 1536 individual arrays. As will be appreciated by those in the art, each microtiter well need not contain an individual array. Composite arrays can include individual arrays that are identical, similar or different. Alternative combinations, where rows, columns or other portions of a microtiter formatted array are the same can be used, for example, in cases where redundancy is desired. As will be appreciated by those in the art, there are a variety of ways to configure a composite array. In addition, in embodiments where random arrays are used, the same population of beads can be added to two different surfaces, resulting in substantially similar but perhaps not identical arrays.

A substrate used in a universal or locus-specific array of the invention can be made from any material that can be modified to contain discrete individual sites and is amenable to at least one detection method. Where arrays of particles are used, a material that is capable of attaching or associating with one or more type of particles can be used. Useful substrates include, but are not limited to, glass; modified glass; functionalized glass; plastics such as acrylics, polystyrene and copolymers of styrene and other materials, polypropylene, polyethylene, polybutylene, polyurethanes, Teflon, or the like; polysaccharides; nylon; nitrocellulose; resins; silica; silica-based materials such as silicon or modified silicon; carbon; metal; inorganic glass; optical fiber bundles, or any of a variety of other polymers. Useful substrates include those that allow optical detection, for example, by being translucent to energy of a desired detection wavelength and/or do not themselves appreciably fluorescese in a desired detection wavelength.

Generally a substrate used for a universal or locus-specific array of the invention has a flat or planar surface. However, other configurations of substrates can be used as well. For example, three dimensional configurations can be used by embedding an array, such as a bead array in a porous material, such as a block of plastic, that allows sample access to the array locations and use of a confocal microscope for detection. Similarly, assay locations can be placed on the inside surface of a tube, for flow-through sample analysis. Exemplary substrates that are useful in the invention include, but are not limited to, optical fiber bundles, or flat planar substrates such as glass, polystyrene or other plastics and acrylics.

The surface of a substrate used for the universal or locus-specific arrays of the invention can include a plurality of individual array locations that are physically separated from each other. For example, physical separation can be due to the presence of assay wells, such as in a microtiter plate. In another example, arrays on the surface of a microscope slide can be separated by a removable seal or gasket. Other barriers that can be used to physically separate array locations include, for example, hydrophobic regions that will deter flow of aqueous solvents or hydrophilic regions that will deter flow of apolar or hydrophobic solvents.

The sites on a universal or locus-specific array can be a pattern such as a regular design or configuration, or the sites can be in a non-patterned distribution. A non-limiting advantage of a regular pattern of sites is that the sites can be conveniently addressed in an X-Y coordinate plane. A pattern in this sense includes a repeating unit cell, such as one that allows a high density of beads on a substrate. An array substrate useful for the universal arrays or locus-specific arrays of the invention can be an optical fiber bundle or array, as is generally described in U.S. Ser. No. 08/944,850, U.S. Pat. No. 6,200,737; WO9840726, and WO9850782, all of which are expressly incorporated herein by reference. Also useful in the invention is a preformed unitary fiber optic array having discrete individual fiber optic strands that are co-axially disposed and joined along their lengths. A distinguishing feature of a preformed unitary fiber optic array compared to other fiber optic formats is that the fibers are not individually physically manipulable; that is, one strand generally cannot be physically separated at any point along its length from another fiber strand.

When particles are used, unique optical signatures can be incorporated into the particles and can be used to identify the chemical functionality or nucleic acid associated with the particle. Exemplary optical signatures include, without limitation, dyes, usually chromophores or fluorophores, entrapped or attached to the beads. Different types of dyes, different ratios of mixtures of dyes, or different concentrations of dyes, or a combination of these differences can be used as optical signatures. Further examples of particles and other supports having detectable signatures that can be used in the invention are described in Cunin et al., Nature Materials 1:39-41 (2002); U.S. Pat. No. 6,023,540 or 6,327,410; or WO9840726, all hereby expressly incorporated by reference.

It should be noted that not all sites of a universal array used to make the locus-specific arrays of the invention need to include a probe or particle. Thus, a universal array can have one or more array locations on the substrate that are empty. An array substrate can also include one or more sites that contain more than one bead or probe. Furthermore, a locus-specific array of the invention can have one or more locations that have not been converted to locus-specific array locations. Such locations are useful, for example, as fiducials to help register multiple images of the same array when compared to each other. These and other fiducials useful in an array of the invention are known in the art as described, for example, in WO 02/12897 and WO 00/47996, hereby expressly incorporated by reference.

Methods of attachment of oligonucleotides to particles or other solid materials are well known in the art of microarrays. Probes can be attached to functional groups on a solid support. Functionalized solid supports can be produced by methods known in the art and, if desired, obtained from any of several commercial suppliers for beads and other supports having surface chemistries that facilitate the attachment of a desired functionality by a user. Exemplary surface chemistries that are useful in the invention include, but are not limited to, amino groups such as aliphatic and aromatic amines, carboxylic acids, aldehydes, amides, chloromethyl groups, hydrazide, hydroxyl groups, sulfonates or sulfates. If desired, a probe can be attached to a solid support via a chemical linker. Such a linker can have characteristics that provide, for example, stable attachment, reversible attachment, sufficient flexibility to allow desired interaction with a given target to be detected, or to avoid undesirable binding reactions. Further exemplary methods that can be used in the invention to attach polymer probes to a solid support are described in Pease et al., Proc. Natl. Acad. Sci. USA 91(11):5022-5026 (1994); Khrapko et al., Mol Biol (Mosk) (USSR) 25:718-730 (1991); Stimpson et al., Proc. Natl. Acad. Sci. USA 92:6379-6383 (1995) or Guo et al., Nucleic Acids Res. 22:5456-5465 (1994), all hereby expressly incorporated by reference.

Probes or particles with associated probes can be attached to a substrate in a non-random or ordered process. For example, using photoactivatible attachment linkers or photoactivatible adhesives or masks, selected sites on an array substrate can be sequentially activated for attachment, such that defined populations of nucleotides, adapter probes or particles are laid down at defined positions in the universal array when exposed to the activated array substrate.

Alternatively, probes or particles with associated probes can be randomly deposited on a substrate and their positions in the array determined by a decoding step. This can be done before, during or after the use of the array to detect target nucleic acids. When the placement of probes is random, a coding or decoding system can be used to localize and/or identify the probes at each location in the array. This can be done in any of a variety of ways, as is described, for example, in U.S. Pat. No. 6,355,431.

As will be appreciated by those in the art, a random array need not necessarily be decoded to be useful for the methods of the invention. Beads or probes can be attached to an array substrate, and a detection assay performed. Array locations that have a positive signal for presence of a capture or extended-capture-probe:target hybrid having a particular sequence can be marked or otherwise identified to distinguish or separate it from other array locations. For example, in applications where beads are labeled with a fluorescent dye, array locations for positive or negative beads can be marked by photobleaching. Further exemplary marks include, but are not limited to, non-fluorescent precursors that are converted to fluorescent form by light activation or photocrosslinking groups which can derivatize a probe or particle with a label or substrate upon irradiation with light of an appropriate wavelength.

The invention can also be used with a liquid array in which particles are aligned in a fluid stream. Individual particles in a liquid array can be identified according to their position in a fluid stream using, for example a flow cytometer, fluorescence activated cell sorter or similar device. In accordance with the invention, a universal liquid array of particles having adapter probe oligonucleotides can be converted to a locus-specific liquid array by modifying the adapter probes to locus-specific capture probes. Exemplary fluid arrays that can be used in the invention include, for example, those described in U.S. Pat. Pub, 2001/0055801A1 or WO0114589A2.

Several levels of redundancy can be built into a locus-specific array used in the invention. Building redundancy into an array can give several non-limiting advantages, including the ability to make quantitative estimates of confidence about the data and substantial increases in sensitivity. As will be appreciated by those in the art, there are at least two types of redundancy that can be built into an array: the use of multiple identical probes or the use of multiple probes directed to the same target, but having different chemical functionalities. For example, for the detection of nucleic acids, sensor redundancy utilizes a plurality of sensor elements such as beads having identical binding ligands such as probes. Target redundancy utilizes sensor elements with different probes to the same target: one probe can span the first 25 bases of a target, a second probe can span the second 25 bases of the target, etc. By building in either or both of these types of redundancy into an array a variety of statistical mathematical analyses can be done for analysis of large data sets. Other methods for decoding with redundant sensor elements and target elements that can be used in the invention are described, for example, in U.S. Pat. No. 6,355,431.

Nucleic acid probes of universal arrays used in the invention can be attached to particles that are arrayed or otherwise spatially distinguished. Exemplary particles include microspheres or beads. However, particles used in the invention need not be spherical. Rather particles having other shapes including, but not limited to, disks, plates, chips, slivers or irregular shapes can be used. In addition, particles used in the invention can be porous, thus increasing the surface area available for attachment or assay of probe-fragment hybrids. Particle sizes can range, for example, from nanometers such as about 100 nm beads, to millimeters, such as about 1 mm beads, with particles of intermediate size such as at most about 0.2 micron, 0.5 micron, 5 micron or 200 microns being useful. The composition of the beads can vary depending, for example, on the application of the invention or the method of synthesis. Suitable bead compositions include, but are not limited to, those used in peptide, nucleic acid and organic moiety synthesis, such as plastics, ceramics, glass, polystyrene, methylstyrene, acrylic polymers, paramagnetic materials, thoria sol, carbon graphite, titanium dioxide, latex or cross-linked dextrans such as Sepharose™, cellulose, nylon, cross-linked micelles or Teflon™. Useful particles are described, for example, in Microsphere Detection Guide from Bangs Laboratories, Fishers Ind.

Those skilled in the art will recognize that particles of other shapes and sizes, such as those set forth above, can be used in place of beads or microspheres.

The sites of a universal or custom array of the invention need not be discrete sites. For example, it is possible to use a uniform surface of adhesive or chemical functionalities, for example, that allows the attachment of particles at any position. That is, the surface of an array substrate can be modified to allow attachment of microspheres at individual sites, whether or not those sites are contiguous or non-contiguous with other sites. Thus, the surface of a substrate can be modified to form discrete sites such that only a single bead is associated with the site or, alternatively, the surface can be modified such that beads end up randomly populating sites in various numbers.

The surface of the substrate can be modified to contain wells, or depressions in the surface of the substrate. This can be done using a variety of techniques, including, but not limited to, photolithography, stamping techniques, molding techniques or microetching techniques. As will be appreciated by those in the art, the technique used will depend on the composition and shape of the substrate. When the substrate for a composite array is a microtiter plate, a molding technique can be utilized to form bead wells in the bottom of the assay wells.

Physical alterations can be made in a surface of a substrate to produce array locations. For example, when the substrate is a fiber optic bundle, the surface of the substrate can be a terminal end of the fiber bundle, as is generally described in U.S. Pat. Nos. 6,023,540 and 6,327,410. Wells can be made in a terminal or distal end of a fiber optic bundle having several individual fibers. Cores of the individual fibers can be etched, with respect to the cladding, such that small wells or depressions are formed at one end of the fibers. The depth of the wells can be altered using different etching conditions to accommodate particles of a particular size or shape. Generally, the microspheres are non-covalently associated in the wells, although the wells can additionally be chemically functionalized for covalent binding of particles. Cross-linking agents can be used, or a physical barrier can be used such as a film or membrane over the particles.

The surface of a substrate can be modified to contain chemically modified sites that are useful for attaching, either-covalently or non-covalently, probes or particles having attached probes. Chemically modified sites in this context include, but are not limited to, the addition of a pattern of chemical functional groups including, for example, amino groups, carboxy groups, oxo groups or thiol groups. Such groups can be used to covalently attach probes or particles that contain corresponding reactive functional groups. Other useful surface modifications include, for example, the addition of a pattern of adhesive that can be used to bind particles; the addition of a pattern of charged groups for the electrostatic attachment of probes or particles; the addition of a pattern of chemical functional groups that render the sites differentially hydrophobic or hydrophilic, such that the addition of similarly hydrophobic or hydrophilic probes or particles under suitable conditions will result in association to the sites on the basis of hydroaffinity.

Once microspheres are generated, they can be added to a substrate to form an array. Arrays can be made, for example, by adding a solution or slurry of the beads to a substrate containing attachment sites for the beads. A carrier solution for the beads can be a pH buffer, aqueous solvent, organic solvent, or mixture. Following exposure of a bead slurry to a substrate, the solvent can be evaporated, and excess beads removed. When non-covalent methods are used to associate beads to an array substrate, beads can be loaded onto the substrate by exposing the substrate to a solution of particles and then applying energy, for example, by agitating or vibrating the mixture. However, static loading can also be used if desired. Methods for loading beads and other particles onto array substrates that can be used in the invention are described, for example, in U.S. Pat. No. 6,355,431.

As described above, universal arrays useful for making locus-specific arrays using the methods of the invention can be comprised of a plurality of adapter probes, each potentially having a different sequence. The chimeric oligonucleotides used can include an adapter-specific portion, having a sequence identical or complementary to an adapter probe sequence, and a locus-specific portion, having sequence identical to or complementary to a target sequence. Complementarity or identity as used in the terms can be perfect or less than perfect such as at least about 99%, 97%, 95% or 90% complementary or identical. Specificity of hybridization can be influenced by percent complementarity, stringency of hybridization conditions, or both. More specifically, higher specificity can be achieved as stringency is increased and/or percent complementarity is increased. Exemplary hybridization conditions are set forth below.

The adapter-specific portion of the chimeric oligonucleotide can be comprised of a portion, which has complementarity to the adapter probe and can form a hybrid with the adapter probe. The locus-specific portion of the chimeric oligonucleotide can be comprised of a locus-specific portion, which has complementarity to a locus-specific sequence, which in turn can be complementary or identical to a locus sequence. Therefore, the locus-specific portion can be complementary or identical to a locus sequence, and can form a hybrid with its complementary locus-specific or locus oligonucleotide.

In embodiments, the chimeric oligonucleotides can be contacted with the adapter probe oligonucleotides under conditions for forming a plurality of chimeric-oligonucleotide:adapter probe oligonucleotide hybrids.

A variety of hybridization conditions known to those of skill in the art may be used for the various hybridization steps in the present invention, including high, moderate and low stringency conditions; see for example Maniatis et al., Molecular Cloning: A Laboratory Manual, 2d Edition, 1989, and Short Protocols in Molecular Biology, ed. Ausubel, et al., hereby incorporated by reference. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Acid Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength and pH. The Tₘ is the temperature (under defined ionic strength, pH and nucleic acid concentration) at which 50% of the probes hybridize to their complement sequence at equilibrium (as the complementary sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g. 10 to 50 nucleotides) and at least about 60° C. for long oligonucleotides (e.g. greater than 50 nucleotides). Stringent conditions may also be achieved with the addition of helix-destabilizing agents such as formamide. The hybridization conditions may also vary when a non-ionic backbone, i.e. PNA is used, as is known in the art. Cross-linking agents may additionally be added after target binding, to cross-link the two strands of the hybridization complex.

Stringency can be controlled by altering a step parameter that is a thermodynamic variable, including, but not limited to, temperature, formamide concentration, salt concentration, chaotropic salt concentration, pH, organic solvent concentration and other parameters known to those of skill in the art.

These parameters may also be used to control non-specific binding, as is generally outlined in U.S. Pat. No. 5,681,697, hereby expressly incorporated by reference. Thus it may be desirable to perform certain steps at higher stringency conditions to reduce non-specific binding.

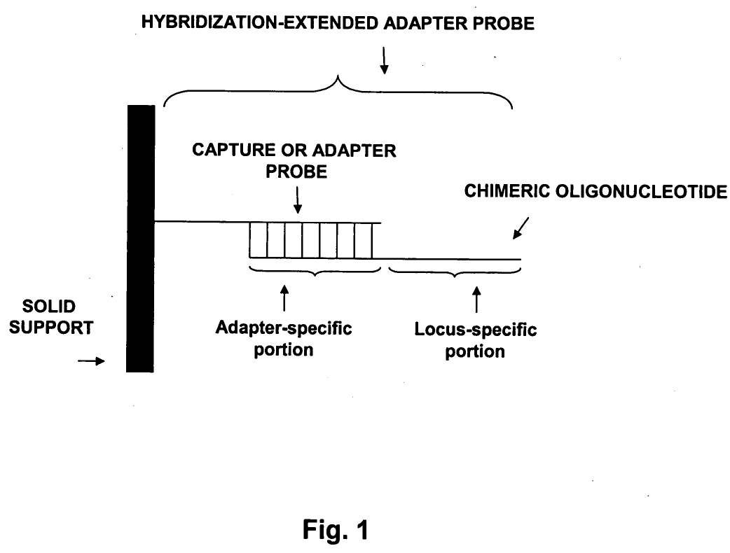

The chimeric oligonucleotide:adapter probe hybrids have single-stranded locus-specific or cLocus (complementary to the locus) portions that can be used to detect target oligonucleotides in a sample. A schematic diagram illustrating this “sandwich” hybrid is shown in FIG. 1.

In embodiments of the invention, the adapter probe:chimeric oligonucleotide hybrid is immobilized. The hybrid can be immobilized by crosslinking the two oligonucleotides using a crosslinking compound. The crosslinking compound may be positioned within a nucleotide sequence of a probe or oligonucleotide, or one oligonucleotide may incorporate the crosslinking compound while the other probe may consist of one or more modified or unmodified purine or pyrimidine nucleoside(s) or derivative(s) which function as reactant for the crosslinking compound. Examples of crosslinking compounds that react with crosslinking compound reactants such as modified or unmodified pyrimidine nucleosides or derivatives are coumarin derivatives including (1) 3-(7-coumarinyl)glycerol; (2) psoralen and its derivatives, such as 8-methoxypsoralen or 5-methoxypsoralen; (3) cis-benzodipyrone and its derivatives; (4) trans-benzodipyrone; and (5) compounds containing fused coumarin-cinnoline ring systems. All of these molecules contain the necessary crosslinking group (an activated double bond) located in an orientation and at a distance to permit crosslinking with a nucleotide. Suitable crosslinking agents include coumarin derivatives containing a basic coumarin (benzopyrone) ring system on which the remainder of the molecule is based. These crosslinking compounds are discussed in detail in U.S. Pat. No. 6,005,093, hereby expressly incorporated by reference. Furthermore, crosslinking services using coumarin-based nucleotide analogs are available (Naxcor, Inc., Menlo Park). Methods for psoralen-based crosslinking are described, for example, in Gunderson et al., Genome Research 8:1142-1153, 1998, hereby expressly incorporated by reference.

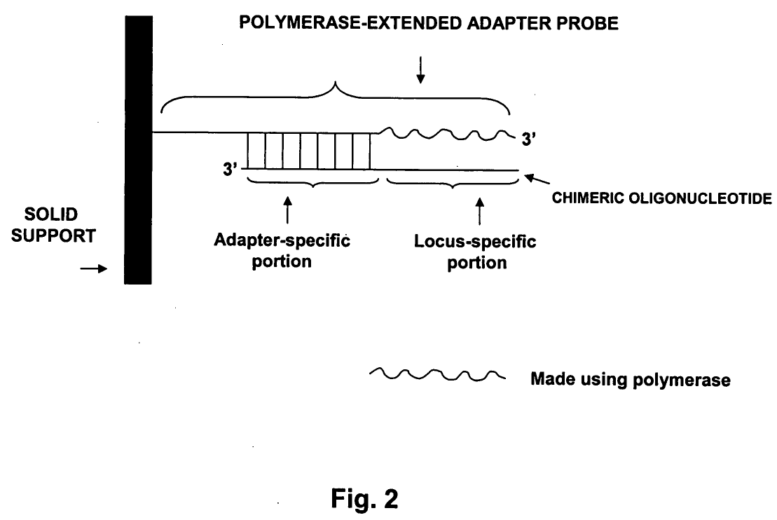

In certain embodiments of the invention, an adapter probe of a universal array can be extended by a polymerase to generate a locus-specific probe. (See FIG. 2) The orientation of the adapter probe oligonucleotide on the universal array can be such that it can be used as a primer for a polymerase, given a suitably hybridized template oligonucleotide, e.g., a chimeric oligonucleotide comprising an adapter-specific portion and a locus-specific portion. In an exemplary embodiment, an adapter probe can be attached to an array location such that the 3′ end of the probe is available for modification by a polymerase. The chimeric oligonucleotide can be hybridized under high stringency to the adapter-probe oligonucleotide, resulting in hybridization of the adapter-specific portion of the chimeric oligonucleotide to the adapter-probe oligonucleotide. The single-stranded locus-specific portion of the chimeric oligonucleotide is thus free to serve as a template for polymerase extension of the 3′ end of the adapter-probe oligonucleotide. The array can be washed following hybridization, and polymerase extension performed directly on the array. Extension results in the production of a locus-specific probe from the adapter probe of the universal array. In other embodiments, the polymerase extension can be performed using as a template a chimeric oligonucleotide comprising an intervening sequence separating the locus-specific portion from the adapter-specific portion of the chimeric oligonucleotide.

Methods for performing polymerase extension are well-known to those of skill in the art, and described, e.g., in Maniatis et al., Molecular Cloning: A Laboratory Manual, 2d Edition, 1989, and Short Protocols in Molecular Biology, ed. Ausubel, et al., hereby expressly incorporated by reference.

As used herein, the term “polymerase” is intended to mean an enzyme that produces a complementary replicate of a nucleic acid molecule using the nucleic acid as a template strand. DNA polymerases bind to the template strand and then move down the template strand adding nucleotides to the free hydroxyl group at the 3′ end of a growing chain of nucleic acid. DNA polymerases synthesize complementary DNA molecules from DNA or RNA templates and RNA polymerases synthesize RNA molecules from DNA templates (transcription). DNA polymerases generally use a short, preexisting RNA or DNA strand, called a primer, to begin chain growth. Examples of polymerases that can be used in methods of this invention are and conditions under which polymerases reaction mixtures can be appled to arrays of the invention are set forth below.

T4 DNA polymerase can be used for primer extension in a method of the invention. For example, pre-extension buffer can be prepared by combining, per 20 μl reaction volume, 10×T4 DNA polymerase buffer (add 2 μl, to a final concentration of 1×); water (87 μl); 10% Tween-20 (1 μl, to a final concentration of 0.1%); and 10 mg/ml BSA (1 μl, to a final concentration of 100 μg/ml). Oligonucleotide extension solutions can be prepared by combining, per 20 μl reaction volume, 10×T4 DNA polymerase buffer (2 μl, to a final concentration of 1×); 25 mM MgCl (4 μl, to a final concentration of 5 mM; 1 mM dNTPs (2 μl to a final concentration of 100 μM); 10 mg/ml BSA (1 μl, to a final concentration of 100 μg/ml); 10% Tween-20 (1 μl, to a final concentration of 0.1%); 100 mM DTT (0.2 μl, to a final concentration of 1 mM); 5 U/μl Klenow enzyme (0.2 μl, a total of 1 U); water (11.2 μl). The reaction can be incubated for 15 minutes at 37° C., for example, in 50 mM HEPES pH 7.5, 50 mM Tris-HCl pH 8.6, or 50 mM glycinate pH 9.7. Another exemplary reaction condition contains 50 mM KCl, 5 mM MgCl₂, 5 mM dithiothreitol (DTT), 0.2 mM of each dNTP, 50 ug/ml BSA, 100 uM random primer (n=6) and 10 units of T4 polymerase incubated at 37° C. for at least one hour.

T7 polymerase can also be used in the methods described herein. Useful reaction conditions include, for example, 40 mM Tris-HCl pH 7.5, 15 mM MgCl₂, 25 mM NaCl, 5 mM DTT, 0.25 mM of each dNTP, and 0.5 to 1 unit of T7 polymerase. Form 1 T7 polymerase and modified T7 polymerase (SEQUENASE™ version 2.0 which lacks the 28 amino acid region Lys118 to Arg 145) can be used. Accordingly, probes can be extended in a method of the invention using a modified T7 polymerase or modified conditions such as those set forth above.

Taq polymerase is another useful enzyme for probe extension. Exemplary conditions include Tris-HCl at about 20 mM, pH of about 7, about 1 to 2 mM MgCl₂, and 0.2 mM of each dNTP. Additionally a stabilizing agent can be added such as glycerol, gelatin, BSA or a non-ionic detergent. Such stabilizing agents can be added to other polymerase-containing reaction mixtures described herein as well. In another embodiment, the Stoffel Fragment, which lacks the N-terminal 289 amino acid residues of Taq polymerase, can be used in a method of the invention.

Those skilled in the art will recognize that the conditions for extension with the various polymerases as set forth above are exemplary. Thus, minor changes that do not substantially alter activity can be made. Furthermore, the conditions can be substantively changed to achieve a desired activity or to suit a particular application of the invention.

The invention can also be carried out with variants of the above-described polymerases, so long as they retain polymerase activity. Exemplary variants include, without limitation, those that have decreased exonuclease activity, increased fidelity, increased stability or increased affinity for nucleoside analogs. Exemplary variants as well as other polymerases that are useful in a method of the invention include, without limitation, bacteriophage phi29 DNA polymerase (U.S. Pat. Nos. 5,198,543 and 5,001,050), exo(−)Bca DNA polymerase (Walker and Linn, Clinical Chemistry 42:1604-1608 (1996)), phage M2 DNA polymerase (Matsumoto et al., Gene 84:247 (1989)), phage phiPRD 1 DNA polymerase (Jung et al., Proc. Natl. Acad. Sci. USA 84:8287 (1987)), exo(−)VENTTM DNA polymerase (Kong et al., J. Biol. Chem. 268.1965-1975 (1993)), T5 DNA polymerase (Chatterjee et al., Gene 97:13-19 (1991)), and PRD1 DNA polymerase (Zhu et al., Biochim. Biophys. Acta. 1219:267-276 (1994)). These references are all hereby expressly incorporated by reference.

A typical array location, such as a bead, can contain a large population of relatively densely packed probe nucleic acids. Following hybridization of target nucleic acids under many conditions only a portion of probes in a detection assay will be occupied with a complementary target. Under such conditions it is possible that densely packed probes will form inter-probe structures that are susceptible to ectopic primer extension. Furthermore, probes having self-complementary sequences can also form structures that are susceptible to ectopic primer extension. Ectopic extension refers to modification of one or both probes in an inter- or intra-probe hybrid during an extension reaction. Ectopic extension can occur irregardless of the presence of a hybridized target to the array. Ectopic extension can be reduced or avoided in a method of the invention using an ectopic extension inhibitor.

An ectopic extension inhibitor useful in the invention can be any agent that is capable of binding to a single-stranded nucleic acid probe, thereby preventing hybridization of the probe to a second probe. Exemplary agents include, but are not limited to single-stranded nucleic acid binding proteins (SSBs), nucleic acids such as those set forth above including nucleic acid analogs or small molecules. Such agents have the general property of preferentially binding to single-stranded nucleic acids over double-stranded nucleic acids irrespective of the nucleotide sequence. Exemplary single-stranded nucleic acid binding proteins that can be used in the invention include, but are not limited to, RecA, Eco SSB, T4 gp32, T7 SSB, N4 SSB, Ad SSB, UP1, and the like and others described, for example, in Chase et al, Ann. Rev. Biochem., 55: 103-36 (1986); Coleman et al, CRC Critical Reviews in Biochemistry, 7(3): 247-289 (1980) and U.S. Pat. No. 5,773,257, all hereby expressly incorporated by reference. Ectopic extension in any of the primer extension assays set forth above can be inhibited using a method of the invention.

An ectopic extension inhibitor can be added under conditions where it coats single-stranded oligonucleotides that have not hybridized to a complementary nucleic acid such as a chimeric oligonucleotide or splint oligonucleotide. The bound inhibitor thus prevents self-annealing and subsequent extension of the single-stranded oligonucleotides. An agent such as a protein that binds to single-stranded probes can be added to a population of probes prior to or during a primer extension reaction, for example, prior to or during an annealing step.

Ectopic expression can also be reduced using one or more blocking oligos. For example, a blocking oligo that is complementary to the 3′ end of a probe can be added under conditions where it will hybridize to probes that have not hybridized to a target nucleic acid. In applications where several probes are present, a plurality of blocking oligos designed to anneal to the 3′ ends of the probes can be added. One or more blocking oligos can be added to a population of probes prior to or during a primer extension reaction, for example, prior to or during an annealing step.

In some embodiments, a probe can be designed with complementary sequence portions capable of forming a hairpin structure that is not capable of being extended under the conditions used for the primer extension step in a primer extension assay. A probe can be designed to have a first sequence region adjacent to the 3′ end of the probe that is complementary to a second sequence region of the probe such that a hairpin forms with a 3′ overhang that is not capable of being extended. The hairpin structure is further designed such that it does not inhibit annealing to target nucleic acids under conditions of the annealing step of a primer extension reaction. For example, two regions of a probe can have complementary sequences that do not substantially anneal at temperatures used during target hybridization, but become annealed to form a hairpin once the temperature is reduced for extension.

Although methods for reducing ectopic extension are exemplified above with respect to arrayed probes, those skilled in the art will recognize that the methods can be similarly applied to extension reactions in other formats such as solution phase reactions or beads spatially separated in fluid phase.

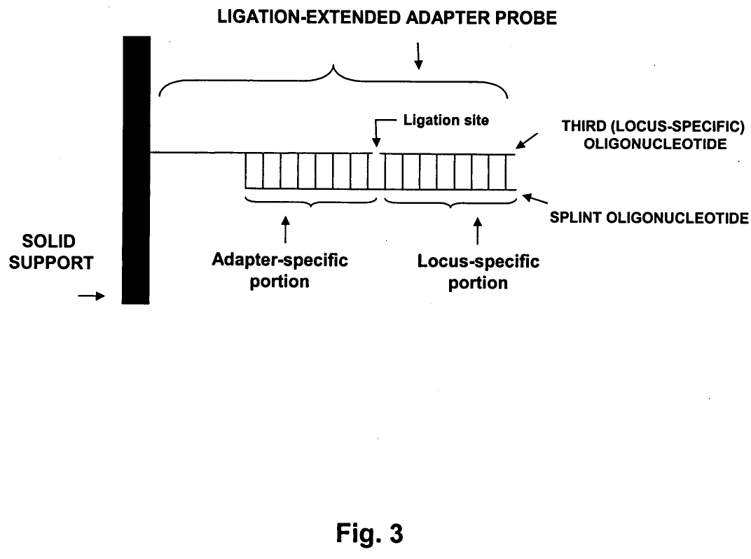

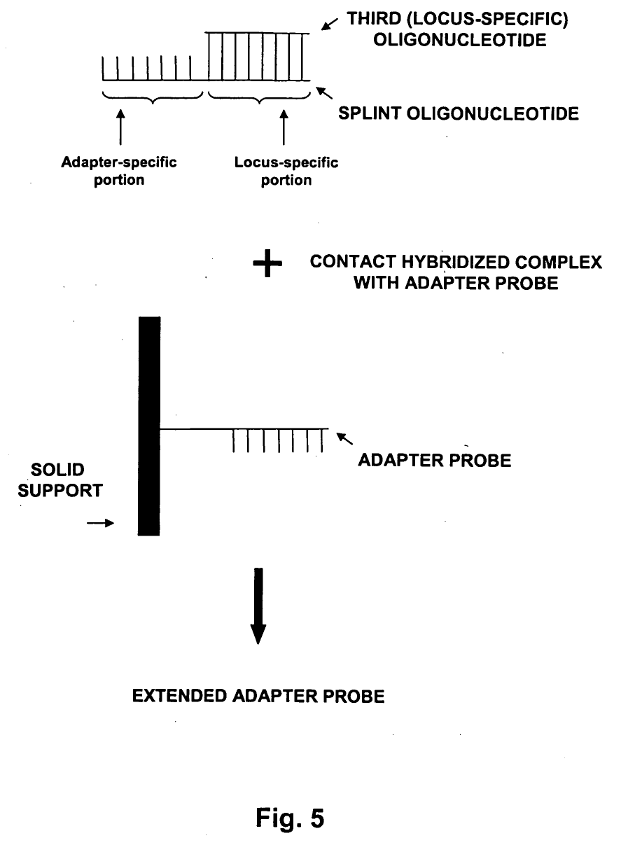

In other embodiments, the locus-specific portion of a locus splint oligonucleotide can hybridize to a third, locus-specific, oligonucleotide, or the second adapter specific portion of an adapter splint oligonucleotide can hybridize to a chimeric oligonucleotide. Hybridization can take place either in solution, and the resulting locus splint oligonucleotide:third oligonucleotide hybrid and/or adapter splint oligonucleotide:chimeric oligonucleotide hybrid subsequently contacted with the capture probe of the array (see FIG. 5). Hybridization of a locus splint oligonucleotide to a third oligonucleotide and/or hybridization of an adapter splint oligonucleotide with a chimeric oligonucleotide can take place in the presence of the adapter probe of the array. An advantage of prehybridizing the splint oligonucleotides to their corresponding locus-specific oligonucleotide and/or chimeric oligonucleotide is that after they are hybridized, the duplex products can be pooled and hybridized “en masse” to the array. Following hybridization, the third oligonucleotide and/or chimeric oligonucleotide can then be ligated, to the end of the adapter probe that is not attached to the array, by means of chemical or enzymatic ligation. A schematic diagram illustrating ligation extension is shown in FIG. 3.

Chemical and enzymatic methods of ligating oligonucleotides are well-known in the art. Chemical ligation methods are generally outlined in U.S. Pat. Nos. 5,616,464 and 5,767,259, both of which are hereby expressly incorporated by reference in their entirety. As with enzymatic ligation, two oligonucleotides, for example, the adapter probe and a third, locus-specific oligonucleotide are utilized. These oligonucleotides are adjacent with regard to a complementary sequence, for example, the splint oligonucleotide.

Enzymatic ligation requires a 5′ phosphate and a 3′ OH at the ligation junction, and a ligase. Enzymatic ligation can be carried out under known conditions for the particular ligase enzyme being used as described, for example, in Sambrook et al., Molecular Cloning: A Laboratory Manual, 3rd edition, Cold Spring Harbor Laboratory, New York (2001) or in Ausubel et al., Current Protocols in Molecular Biology, John Wiley and Sons, Baltimore, Md. (1998), hereby expressly incorporated by reference. Chemical ligation can employ different combinations of 3′ OH/phosphate and 5′ OH/phosphate combinations to effect ligation, dependent upon the particular chemistry. For instance, carbodiimide-mediated ligation prefers both a 3′ and 5′ phosphate for optimal coupling, but ligation also occurs with either a 3′ or 5′ phosphate. Cyanogen bromide-mediated chemical ligation is most efficient with a 3′ phosphate and a 5′ OH group at the junction, but can also use other combinations (see Shabarova, Z A, “Chemical development in the design of oligonucleotide probes for binding to DNA and RNA,” Biochimie 70:1323-1334, 1988, hereby expressly incorporated by reference).

Chemical ligation offers several potential advantages over enzymatic ligation including better accessibility to all ligation junction sites, for example, in cases where the ligase enzyme might be sterically hindered, and selection of full-length 3′ OH probes. Another advantage of chemical ligation is its low cost. A 350 ml chemical ligation reaction costs about $12.00 (50 mM EDC). In contast, a similar enzymatic ligation costs about $840 (using 1 U T4 DNA ligase). A 350 ml volume can process about 27 slides in bulk. For typical array-based ligation conditions, see Gunderson, et al., Genome Research (1998), hereby expressly incorporated by reference.

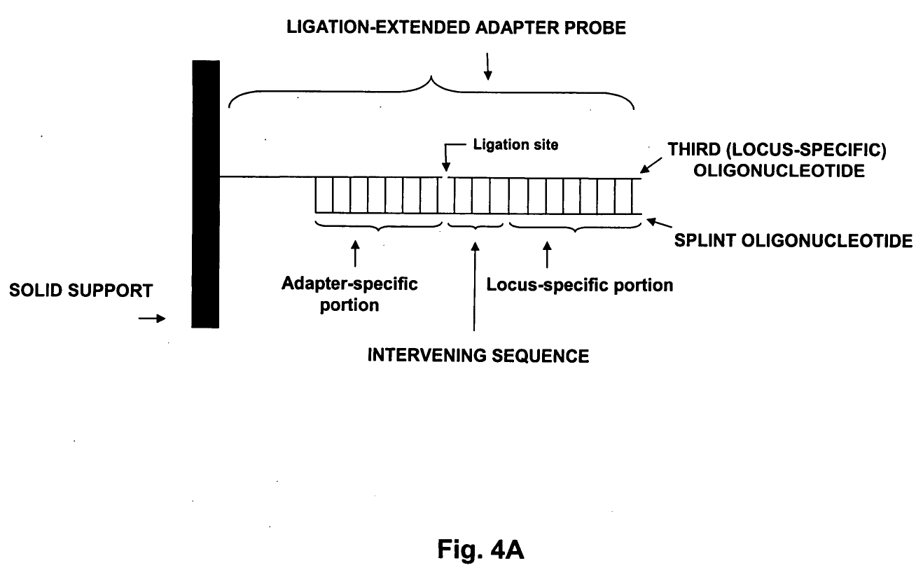

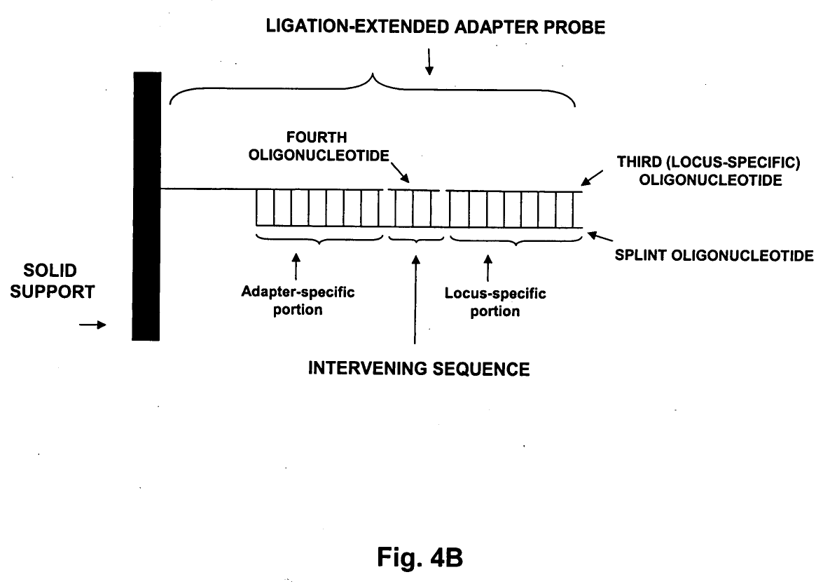

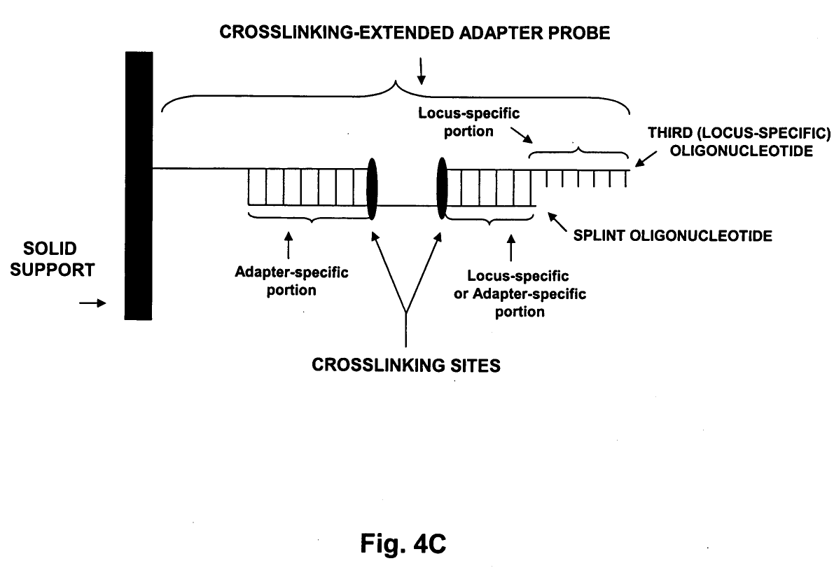

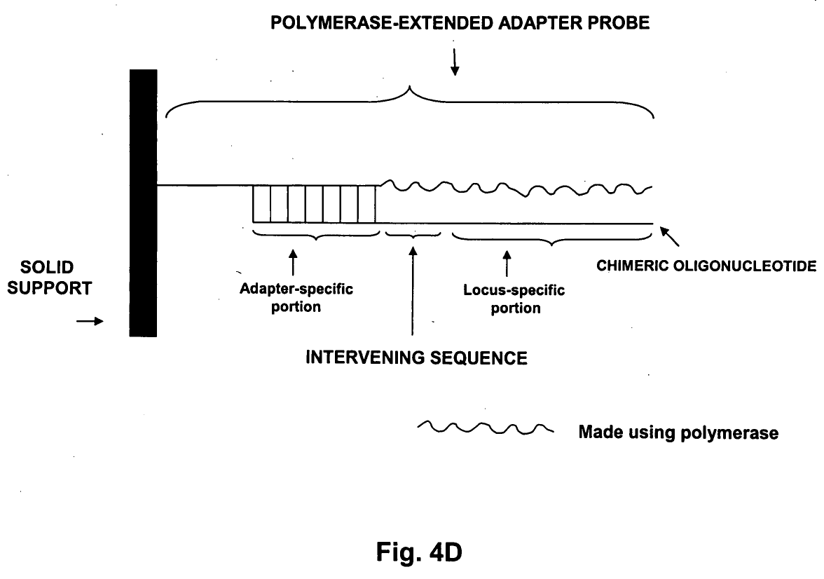

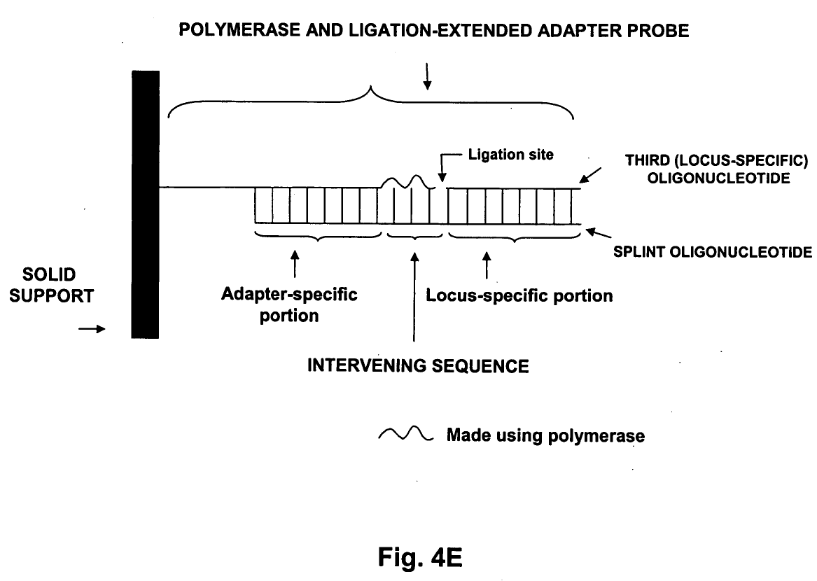

In embodiments of the present invention, the splint oligonucleotide comprises a locus specific portion or a second adapter portion separated from the portion specific for the adapteron a solid support by an intervening sequence. (See FIG. 4).

In certain embodiments, the gap is not filled in, rather, the splint oligonucleotide is crosslinked at separate locations to both the adapter probe and a locus specific oligonucleotide, e.g., a third oligonucleotide consisting of a locus specific oligonucleotide or a chimeric oligonucleotide to generate a locus-specific probe. In other embodiments of the invention, the locus specific oligonucleotide is a chimeric oligonucleotide which also comprises a portion complementary to the intervening sequence. The additional sequence in the locus-specific oligonucleotide results in adjacent positioning of the locus specific oligonucleotide and the adapter probe hybridized to the chimeric or splint oligonucleotide. The locus specfic oligonucleotide and each respective adapter probe can be ligated to form the extended adapter probe oligonucleotide.

In other embodiments, a fourth oligonucleotide comprising a portion complementary to the intervening sequence can be hybridized to the chimeric or splint oligonucleotide. One end of the fourth oligonucleotide is ligated to the adapter probe, while the other end is ligated to the third oligonucleotide or chimeric oligonucleotide.

In other embodiments of the invention, hybridization of the locus-specific oligonucleotide does not occur immediately adjacent to the adapter probe regardless of the presence of an intervening sequence in the chimeric oligonucleotide. This results in a single-stranded gap which must be filled in prior to ligation. In methods involving ligation of the adapter probe to the locus-specific oligonucleotide when the splint oligonucleotide comprises an intervening sequence between the first adapter-specific and second portions, the gap left can be filled in prior to ligation using methods well-known and described in the art employing a polymerase. In these embodiments, polymerase extension can be used to fill in the resulting “gap” in the adapter probe strand of the hybridization complex by synthesizing the complement of the intervening sequence in addition to that of the adapter or locus-specific sequences which are part of the gap. In other embodiments, polymerase extension can be used to fill in any gap between the 3′ end of the adapter probe and the 5′ end of the third oligonucleotide or chimeric oligonucleotide, whether or not there is an intervening sequence (FIG. 4E). The polymerase can utilize the 3′ OH of the adapter probe as a primer to extend up to the 5′ end of the locus-specific oligonucleotide. The polymerase-extended adapter probe can then be ligated to the locus-specific oligonucleotide to further extend the adapter probe.

Following polymerase extension of the adapter probe or ligation of the adapter probe to a third or fourth oligonucleotide, or chimeric oligonucleotide, the splint oligonucleotide can be denatured and removed from the hybrid structure as desired, for example, to facilitate use of the locus-specific array for detection purposes. Denaturation can be accomplished by utilizing a thermal step, generally by raising the temperature of the reaction to about 95° C., although pH changes and other techniques may also be used. Chemical means of denaturation include incubation in 0.1 N NaOH, or 95% formamide, or similar denaturants.

Methods of selecting appropriate adapter-probe, locus-specific and intervening sequences, as well as optimum lengths for the oligonucleotides of the invention are known to those of skill in the art. Probes can be selected according to complementarity with desired target sequences in a test sample and absence of cross-hybridization with other sequences in the test sample. Well known sequence comparison algorithms can be used including, for example, BLAST (Altschul et al., J. Mol. Biol. 215:403-410 (1990)) or FASTA (Pearson and Lipman, Proc Natl Acad. Sci. USA 85:24442448 (1998)), hereby expressly incorporated by reference.

Probes useful in the invention or portions thereof can be any length desired for a particular application. Thus, adapters, adapter-specific portions, locus-specific portions, chimeric oligonucleotides, splint oligonucleotides and other oligonucleotides can have lengths, for example, of at least 6, 8, 10, 15, 20, 25, 30, 40, 50, 60, 70 or more nucleotides. Exemplary ranges include, but are not limited 6 nucleotides up to 100 nucleotides, with a particularly useful range being from 12-25 bases, from 15 nucleotides to 100 nucleotides or 20-70 nucleotides.

The probes of the present invention can be synthesized by methods commonly practiced by those of skill in the art. For example, polymer probes such as nucleic acids or peptides can be synthesized by sequential addition of monomer units directly on a solid support used in an array such as a bead or slide surface. Methods known in the art for synthesis of a variety of different chemical compounds on solid supports can be used in the invention, such as methods for solid phase synthesis of peptides, organic moieties, and nucleic acids. Methods for synthesizing immobilized polymers are described, for example, in U.S. Pat. Nos. 6,416,952 and 6,600,031, hereby expressly incorporated by reference. Methods for synthesizing polymers discussed in these patents include approaches involving removal and addition of photosensitive protecting agents for adding particular reagents or compounds to regions of a substrate. Alternatively probes can be synthesized first, and then covalently attached to a solid support, as described, for example, in U.S. Pat. No. 6,339,147 or WO 02/10431A2, hereby expressly incorporated by reference. Another useful attachment chemistry includes reaction of a cyanuric chloride derivitized surface with hydrazine to yield hydrazine triazine followed by addition of benzaldehyde oligonucleotides to the triazine resulting in immobilization of the oligonucleotides on the surface.

As will be appreciated by those in the art, the nucleic acid analogs described herein may be used in the arrays and methods of the present invention. In addition, mixtures of naturally occurring nucleic acids and analogs can be made. Alternatively, mixtures of different nucleic acid analogs, and mixtures of naturally occuring nucleic acids and analogs can be made.

The nucleic acid may be DNA, RNA or a hybrid, where the nucleic acid contains any combination of deoxyribo- and ribo-nucleotides, and any combination of bases, including uracil, adenine, thymine, cytosine, guanine, inosine, xanthanine, hypoxanthanine, isocytosine, isoguanine, etc. Use of isocytosine and isoguanine in nucleic acids designed to be complementary to other probes, rather than target sequences, reduces non-specific hybridization, as is generally described in U.S. Pat. No. 5,681,702, hereby expressly incorporated by reference. As used herein, the term “nucleoside” includes nucleotides as well as nucleoside and nucleotide analogs, and modified nucleosides such as amino-modified nucleosides. In addition, “nucleoside” includes non-naturally occuring analog structures. Thus for example the individual units of a peptide nucleic acid, each containing a base, are referred to herein as a nucleoside.

A further example of a nucleic acid with an analog structure that is useful in the invention is a peptide nucleic acid (PNA). The backbone of a PNA is substantially non-ionic under neutral conditions, in contrast to the highly charged phosphodiester backbone of naturally occurring nucleic acids. This provides two non-limiting advantages. First, the PNA backbone exhibits improved hybridization kinetics. Secondly, PNAs have larger changes in the melting temperature (Tₘ) for mismatched versus perfectly matched basepairs. DNA and RNA typically exhibit a 2-4° C. drop in Tₘ for an internal mismatch. With the non-ionic PNA backbone, the drop is closer to 7-9° C. This can provide for better sequence discrimination. Similarly, due to their non-ionic nature, hybridization of the bases attached to these backbones is relatively insensitive to salt concentration.

A nucleic acid useful in the invention can contain a non-natural sugar moiety in the backbone. Exemplary sugar modifications include but are not limited to 2′ modifications such as addition of halogen, alkyl, substituted alkyl, allcaryl, arallcyl, O-allcaryl or O-aralkyl, SH, SCH3, OCN, Cl, Br, CN, CF3, OCF3, SOCH3, SO2 CH3, ONO2, NO2, N3, NH2, heterocycloallcyl, heterocycloallcaryl, aminoallcylamino, polyallcylamino, substituted silyl, and the like. Similar modifications can also be made at other positions on the sugar, particularly the 3′ position of the sugar on the 3′ terminal nucleotide or in 2′-5′ linked oligonucleotides and the 5′ position of 5′ terminal nucleotide.

A nucleic acid used in the invention can also include native or non-native bases. In this regard a native deoxyribonucleic acid can have one or more bases selected from the group consisting of adenine, thymine, cytosine or guanine and a ribonucleic acid can have one or more bases selected from the group consisting of uracil, adenine, cytosine or guanine. Exemplary non-native bases that can be included in a nucleic acid, whether having a native backbone or analog structure, include, without limitation, inosine, xanthanine, hypoxanthanine, isocytosine, isoguanine, 5-methylcytosine, 5-hydroxymethyl cytosine, 2-aminoadenine, 6-methyl adenine, 6-methyl guanine, 2-propyl guanine, 2-propyl adenine, 2-thioLiracil, 2-thiothymine, 2-thiocytosine, 15-halouracil, 15-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. A particular embodiment can utilize isocytosine and isoguanine in a nucleic acid in order to reduce non-specific hybridization, as generally described in U.S. Pat. No. 5,681,702, hereby expressly incorporated by reference.

A non-native base used in a nucleic acid of the invention can have universal base pairing activity, wherein it is capable of base pairing with any other naturally occurring base. Exemplary bases having universal base pairing activity include 3-nitropyrrole and 5-nitroindole. Other bases that can be used include those that have base pairing activity with a subset of the naturally occurring bases such as inosine which basepairs with cytosine, adenine or uracil.

A nucleic acid having a modified or analog structure can be used in the invention, for example, to facilitate the addition of labels, or to increase the stability or half-life of the molecule under conditions of extension, ligation, detection or other conditions used in accordance with the invention. As will be appreciated by those skilled in the art, one or more of the above-described nucleic acids can be used in the present invention, including, for example, as a mixture including molecules with native or analog structures.

A nucleic acid useful in the invention can include a detection moiety. A detection moiety can be a primary label that is directly detectable or secondary label that can be indirectly detected, for example, via direct or indirect interaction with a primary label. Exemplary primary labels include, without limitation, an isotopic label such as a naturally non-abundant radioactive or heavy isotope; chromophore; luminophore; fluorophore; calorimetric agent; magnetic substance; electron-rich material such as a metal; electrochemiluminescent label such as Ru(bpy)32+; or moiety that can be detected based on a nuclear magnetic, paramagnetic, electrical, charge to mass, or thermal characteristic. Fluorophores that are useful in the invention include, for example, fluorescent lanthanide complexes, including those of Europium and Terbium, fluorescein, rhodamine, tetramethylrhodamine, eosin, erythrosin, coumarin, methyl-coumarins, pyrene, Malacite green, Cy3, Cy5, stilbene, Lucifer Yellow, Cascade Blue™, Texas Red, alexa dyes, phycoerythin, bodipy, and others known in the art such as those described in Haugland, Molecular Probes Handbook, (Eugene, Oreg.) 6th Edition; The Synthegen catalog (Houston, Tex.), Lakowicz, Principles of Fluorescence Spectroscopy, 2nd Ed., Plenum Press New York (1999), or WO 98/59066, all of which are hereby expressly incorporated by reference. Labels can also include enzymes such as horseradish peroxidase or alkaline phosphatase or particles such as magnetic particles or optically encoded nanoparticles.

Binding moieties can be used as secondary labels. A binding moiety can be attached to a nucleic acid to allow detection or isolation of the target nucleic acid or removal of chimeric oligonucleotides via specific affinity for a receptor. Specific affinity between two binding partners is understood to mean preferential binding of one partner to another compared to binding of the partner to other components or contaminants in the system. Binding partners that are specifically bound typically remain bound under the detection or separation conditions described herein, including wash steps to remove non-specific binding. Depending upon the particular binding conditions used, the dissociation constants of the pair can be, for example, less than about 10⁻⁴, 10⁻⁵, 10⁻⁶, 10⁻⁷, 10⁻⁸, 10⁻⁹, 10⁻¹⁰, 10⁻¹¹ or 10⁻¹² M⁻¹.

Pairs of binding moieties and receptors that can be used in the invention include, without limitation, antigen and immunoglobulin or active fragments thereof, such as FAbs; immunoglobulin and immunoglobulin (or active fragments, respectively); avidin and biotin, or analogs thereof having specificity for avidin such as imino-biotin; streptavidin and biotin, or analogs thereof having specificity for streptavidin such as imino-biotin; carbohydrates and lectins; and other known proteins and their ligands. It will be understood that either partner in the above-described pairs can be attached to a nucleic acid and detected or isolated based on binding to the respective partner. It will be further understood that several moieties that can be attached to a nucleic acid can function as both primary and secondary labels in a method of the invention. For example, strepatvidin-phycoerythrin can be detected as a primary label due to fluorescence from the phycoerythrin moiety or it can be detected as a secondary label due to its affinity for anti-streptavidin antibodies.

Chemically modifiable moieties can serve as secondary labels. Labels having reactive functional groups can be incorporated into a nucleic acid, and the functional group can be subsequently covalently reacted with a primary label. Suitable functional groups include, but are not limited to, amino groups, carboxy groups, maleimide groups, oxo groups and thiol groups.

Binding moieties can be useful for attaching oligonucleotides to solid surfaces or array components; separating oligonucleotides from other components of a synthetic reaction; concentrating oligonucleotides, or detecting target oligonucleotides when bound to capture probes on an array.

Methods known to those of skill in the art can be used to attach a binding moiety, detection moiety or other useful moiety to an oligonucleotide of this invention, including, for example, a target nucleic acid, adapter probe, chimeric oligonucleotide, splint oligonucleotide, or locus specific oligonucleotide. For example, a primer used to amplify a nucleic acid can include the moiety attached to a base, ribose, phosphate, or analogous structure in a nucleic acid or analog thereof. A moiety can be incorporated using modified nucleosides that are added, for example, to a growing nucleotide strand during amplification or synthesis steps. As set forth below, addition of a detection moiety can also be added during a detection step to indicate interaction of a target oligonucleotide with a probe oligonucleotide on an array. Nucleosides can be modified, for example, at the base or the ribose, or analogous structures in a nucleic acid analog.

An oligonucleotide useful in the invention, such as a locus-specific or adapter probe, can have a structure that is resistant to modification. For example, a probe can lack a 3′ OH group or have a 3′ cap moiety, thereby being inert to modification with a polymerase. A probe can include a detectable label including, without limitation, one or more of the primary or secondary nucleic acid labels set forth above. Alternatively, detection can be based on an intrinsic characteristic of the probe, fragment or hybrid such that labeling is not required. Examples of intrinsic characteristics that can be detected include, but are not limited to, mass, electrical conductivity, energy absorbance, fluorescence or the like.

In embodiments of the invention, the chimeric oligonucleotides can be oriented in the 5′ to 3′ direction from the adapter-specific portion to the locus-specific portion. It will be understood by those of skill in the art that in a nucleic acid polymer, a backbone chain is formed by phosphate linkages between the 5′ carbon of one nucleoside sugar and the 3′ carbon of the adjacent nucleoside sugar. The term “5′ end” of a nucleic acid molecule commonly refers to the end of the nucleic acid chain at which the sugar of the terminal nucleoside has a 5′ carbon group that is not linked to another sugar. The term “3′ end” commonly refers to the end of the molecule at which the sugar of the terminal nucleoside has a free 3′ carbon group that is not linked to another sugar.

Thus, the adapter probe can be oriented in the 3′ to 5′ direction from the attached end to the free end of the probe. Alternatively, and as necessary, the probes can be oriented in the opposite direction. One of skill in the art will understand that the orientation of the oligonucleotides can differ depending upon the nature of the analysis being performed.

In embodiments of the invention, the chimeric oligonucleotides are purified prior to being hybridized to the adapter probes of the universal array. Methods for oligonucleotide purification are well-known to those of skill in the art, and include, for example, PAGE-purification, HPLC purification, cartridge purification, and affinity purification and others such as those described in Sambrook et al., Molecular Cloning: A Laboratory Manual, 3rd edition, Cold Spring Harbor Laboratory, New York (2001) or in Ausubel et al., Current Protocols in Molecular Biology, John Wiley and Sons, Baltimore, Md. (1998), hereby expressly incorporated by reference.

The invention includes methods for using the arrays of the invention to detect a plurality of loci. Typically, target oligonucleotides are detected on a locus-specific array by virtue of their complementarity to locus-specific assay locations. As will be appreciated by those in the art, the sample solution containing the target oligonucleotides may comprise any number of things, including, but not limited to, bodily fluids (including, but not limited to, blood, urine, serum, lymph, saliva, anal and vaginal secretions, perspiration and semen, of virtually any organism, with mammalian samples being preferred and human samples being particularly preferred); environmental samples (including, but not limited to, air, agricultural, water and soil samples); biological warfare agent samples; research samples; purified samples, such as purified genomic DNA, RNA, proteins, etc.; raw samples (bacteria, virus, genomic DNA, etc.; as will be appreciated by those in the art, virtually any experimental manipulation may have been done on the sample). A sample useful in the invention can also be a sub-fraction of those sample listed above.

As described above, the target oligonucleotide can be a portion of a gene, a regulatory sequence, genomic DNA, cDNA, RNA including mRNA and rRNA, or others. The target oligonucleotide sequence may be a target sequence from a sample, or a secondary target such as a product of a reaction such as a detection sequence from an invasive cleavage reaction, a ligated probe from an OLA reaction, an extended probe from a PCR reaction, etc. A target sequence from a sample can be amplified to produce a secondary target that is detected. Alternatively, amplification can be done using a signal probe that is amplified, again producing a secondary target that is detected. The target sequence may be any length, with the understanding that longer sequences are more specific. As will be appreciated by those in the art, the complementary target sequence may take many forms. For example, it may be contained within a larger nucleic acid sequence, i.e. all or part of a gene or mRNA, or a restriction fragment of a plasmid or genomic DNA, among others. Probes are made to hybridize to target sequences to determine the presence or absence of the target sequence in a sample. The target sequence may also be comprised of different target domains; for example, in “sandwich” type assays, a first target domain of the sample target sequence can hybridize to a capture probe, or it can hybridize to a portion of a capture probe extended by polymerase or ligation to a locus-specific oligonucleotide. A second target domain can hybridize to a portion of a different capture probe. In addition, the target domains may be adjacent (i.e. contiguous) or separated.

The target sequence can be prepared using known techniques. For example, the sample can be treated to lyse the cells by methods known to those of skill in the art, e.g., using lysis buffers, sonication, electroporation, with purification occuring as needed, as will be appreciated by those in the art. In addition, the reactions outlined herein may be accomplished in a variety of ways, as will be appreciated by those in the art. Components of the reaction may be added simultaneously, or sequentially, in any order. In addition, the reaction may include a variety of other reagents which may be included in the assays. These include reagents like salts, buffers, neutral proteins, e.g. albumin, detergents, etc., which may be used to facilitate optimal hybridization and detection, and/or reduce non-specific or background interactions. Also reagents that otherwise improve the efficiency of the assay, such as protease inhibitors, nuclease inhibitors, anti-microbial agents, etc., may be used, depending on the sample preparation methods and purity of the target.

Double-stranded target nucleic acids can be denatured by methods known in the art to render them single-stranded so as to permit hybridization of the primers and other probes of the invention. Denaturation can be accomplished by utilizing a thermal step, generally by raising the temperature of the reaction to about 95° C., although pH changes and other techniques may also be used.

Target oligonucleotides can be hybridized to the probes of the locus-specific array under conditions that can be determined by one of skill in the art using general principles of nucleic acid hybridization. Examples of hybridization conditions are described above in relation to formation of the chimeric oligonucleotide:adapter-probe hybrid of the invention.

Detection of the target oligonucleotides hybridized to the locus-specific array can be carried out by a variety of methods known in the art. Target oligonucleotides can include a detection moiety as previously described, and these detection moieties measured by methods known in the art. Methods of detecting molecules on an array that can be used in the invention are set forth below and/or described in the art, for example, in U.S. Pat. Nos. 6,597,000 and 6,650,411, hereby expressly incorporated by reference.

Depending upon the particular application of the invention, target oligonucleotides can be detected using a direct detection technique, or alternatively an amplification-based technique. Direct detection techniques include those in which the level of nucleic acids in probe-fragment hybrids provides the detected signal. For example, in the case of a hybrid formed at a particular array location, the signal from the location arising from the captured hybrid or its component nucleic acids can be detected without amplifying the hybrid or its component nucleic acids. Alternatively, detection can include amplification of the probe or target oligonucleotide or both to increase the level of nucleic acid that is detected. As set forth below in the context of various exemplary detection techniques, a probe nucleic acid, target oligonucleotide or both can be labeled. Furthermore, nucleic acids in a probe-target hybrid can be labeled prior to, during or after hybrid formation and analysis based on detection of such labels.

Generally, detection, whether direct or based on an amplification technique, can be achieved by methods that perceive properties that are intrinsic to nucleic acids or their associated labels. Useful properties include, for example, those that can be used to distinguish nucleic acids having target sequences such as typable loci from those lacking the target sequence. Such detected properties can be used to distinguish different nucleic acids alone or in combination with other methods such as attachment to discrete locations of a detection array. Exemplary properties upon which detection can be based include, but are not limited to, mass, electrical conductivity, energy absorbance, fluorescence or the like.

Detection of fluorescence can be carried out by irradiating a nucleic acid or its label with an excitatory wavelength of radiation and detecting radiation emitted from a fluorophore therein by methods known in the art and described for example in Lakowicz, Principles of Fluorescence Spectroscopy, 2nd Ed., Plenum Press New York (1999), hereby expressly incorporated by reference. A fluorophore can be detected based on any of a variety of fluorescence phenomena including, for example, emission wavelength, excitation wavelength, fluorescence resonance energy transfer (FRET) intensity, quenching, anisotropy or lifetime. FRET can be used to identify hybridization between a first polynucleotide attached to a donor fluorophore and a second polynucleotide attached to an acceptor fluorophore due to transfer of energy from the excited donor to the acceptor. Thus, hybridization can be detected as a shift in wavelength caused by reduction of donor emission and appearance of acceptor emission for the hybrid. In addition, fluorescence recovery after photobleaching (FRAP) can be used to identify hybridization according to the increase in fluorescence occurring at a previously photobleached array location due to binding of a fluorescently-labeled target polynucleotide.

Other detection techniques that can be used to perceive or identify nucleic acids having typable loci include, for example, mass spectrometry, electrophoresis or capillary electrophoresis which can be used to perceive a nucleic acid based on its mass; surface plasmon resonance which can be used to perceive a nucleic acid based on binding to a surface immobilized complementary sequence; absorbance spectroscopy which can be used to perceive a nucleic acid based on the wavelength of the energy it absorbs; calorimetry which can be used to perceive a nucleic acid based on changes in temperature of its environment due to binding to a complementary sequence; electrical conductance or impedence which can be used to perceive a nucleic acid based on changes in its electrical properties or in the electrical properties of its environment, magnetic resonance which can be used to perceive a nucleic acid based on presence of magnetic nuclei, or other known analytic spectroscopic or chromatographic techniques.

In particular embodiments, target sequences of probe-target hybrids can be detected based on the presence of the probe, fragment or both in the hybrid, without subsequent modification of the hybrid species. For example, a pre-labeled fragment having a particular target sequence can be identified based on presence of the label at a particular array location where a nucleic acid complement of the target sequence resides.

In a particular embodiment, arrayed nucleic acid probes can be modified while hybridized to target oligonucleotides for detection. Such embodiments, include, for example, those utilizing ASPE, SBE, oligonucleotide ligation amplification (OLA), extension ligation (GoldenGate™), invader technology, probe cleavage or pyrosequencing as described in U.S. Pat. No. 6,355,431 B1, U.S. Ser. No. 10/177,727, both of which are hereby expressly incorporated by reference and/or below. Thus, the invention can be carried out in a mode wherein an immobilized probe is modified instead of a target oligonucleotide by a probe. In such embodiments an adapter probe can be modified to convert it from a universal adapter probe to a locus-specific adapter probe and in a detection step further modified to indicate presence of a target sequence in a test sample applied to the locus specific probe. Alternatively, detection can include modification of target oligonucleotides while hybridized to probes. Exemplary modifications include those that are catalyzed by an enzyme such as a polymerase. A useful modification can be incorporation of one or more nucleotides or nucleotide analogs to a primer hybridized to a template strand, wherein the primer can be either the probe or target oligonucleotide in a probe-target hybrid. Such a modification can include replication of all or part of a primed template. Modification leading to replication of only a part of a template probe or target oligonucleotide will be understood to be detection without amplification of the template since the template is not replicated along its full length.

Extension assays are useful for detection of target sequences. Extension assays are generally carried out by modifying the 3′ end of a first nucleic acid when hybridized to a second nucleic acid. The second nucleic acid can act as a template directing the type of modification, for example, by base pairing interactions that occur during polymerase-based extension of the first nucleic acid to incorporate one or more nucleotides. Polymerase extension assays are particularly useful, for example, due to the relative high-fidelity of polymerases and their relative ease of implementation. Extension assays can be carried out to modify nucleic acid probes that have free 3′ ends, for example, when bound to a substrate such as an array. Exemplary approaches that can be used include, for example, allele-specific primer extension (ASPE), single base extension (SBE), or pyrosequencing.

Briefly, SBE utilizes an extension probe that hybridizes to a target oligonucleotide at a location that is proximal or adjacent to a detection position, the detection position being indicative of a particular target sequence. A polymerase can be used to extend the 3′ end of the probe with a nucleotide analog labeled with a detection label such as those described previously herein. Based on the fidelity of the enzyme, a nucleotide is only incorporated into the extension probe if it is complementary to the detection position in the target genome fragment. If desired, the nucleotide can be derivatized such that no further extensions can occur, and thus only a single nucleotide is added. The presence of the labeled nucleotide in the extended probe can be detected, for example, at a particular location in an array and the added nucleotide identified to determine the identity of the target sequence. SBE can be carried out under known conditions such as those described in U.S. patent application Ser. No. 09/425,633, hereby expressly incorporated by reference. A labeled nucleotide can be detected using methods such as those set forth above or described elsewhere such as Syvanen et al., Genomics 8:684-692 (1990); Syvanen et al., Human Mutation 3:172-179 (1994); U.S. Pat. Nos. 5,846,710 and 5,888,819; Pastinen et al., Genomics Res. 7(6):606-614 (1997), all hereby expressly incorporated by reference.

A nucleotide analog useful for SBE detection can include a dideoxynucleoside-triphosphate (also called deoxynucleotides or ddNTPs, i.e. ddATP, ddTTP, ddCTP and ddGTP), or other nucleotide analogs that are derivatized to be chain terminating. The use of labeled chain terminating nucleotides is useful, for example, in reactions having more than one type of dNTP present so as to prevent false positives due to extension beyond the detection position. Exemplary analogs are dideoxy-triphosphate nucleotides (ddNTPs) or acyclo terminators (Perkin Elmer, Foster City, Calif.). Generally, a set of nucleotides comprising ddATP, ddCTP, ddGTP and ddTTP can be used, at least one of which includes a label. If desired for a particular application, a set of nucleotides in which all four are labeled can be used. The labels can all be the same or, alternatively, different nucleotide types can have different labels. As will be appreciated by those in the art, any number of nucleotides or analogs thereof can be added to a primer, as long as a polymerase enzyme incorporates a particular nucleotide of interest at an interrogation position that is indicative of a typable locus.

The determination of the base at the detection position can proceed in any of several ways. In a particular embodiment, a mixed reaction can be run with two, three or four different nucleotides, each with a different label. In this embodiment, the label on the probe can be distinguished from non-incorporated labels to determine which nucleotide has been incorporated into the probe. Alternatively, discrete reactions can be run each with a different labeled nucleotide. This can be done either by using a single substrate bound probe and sequential reactions, or by exposing the same reaction to multiple substrate-bound probes. For example, dATP can be added to a probe-fragment hybrid, and the generation of a signal evaluated; the dATP can be removed and dTTP added, etc. Alternatively, four arrays can be used; the first is reacted with dATP, the second with dTTP, etc., and the presence or absence of a signal evaluated in each array.

ASPE is an extension assay that utilizes extension probes that differ in nucleotide composition at their 3′ end. Briefly, ASPE can be carried out by hybridizing a target oligonucleotide to an extension probe having a 3′ sequence portion that is complementary to a detection position and a 5′ portion that is complementary to a sequence that is adjacent to the detection position. Template-directed modification of the 3′ portion of the probe, for example, by addition of a labeled nucleotide by a polymerase yields a labeled extension product, but only if the template includes the target sequence. The presence of such a labeled primer-extension product can then be detected, for example, based on its location in an array to indicate the presence of a particular typable locus.

In particular embodiments, ASPE can be carried out with multiple extension probes that have similar 5′ ends such that they anneal adjacent to the same detection position in a target genome fragment, but different 3′ ends, such that only probes having a 3′ end that complements the detection position are modified by a polymerase. A probe having a 3′ terminal base that is complementary to a particular detection position is referred to as a perfect match (PM) probe for the position, whereas probes that have a 3′ terminal mismatch base and are not capable of being extended in an ASPE reaction are mismatch (MM) probes for the position. The presence of the labeled nucleotide in the PM probe can be detected and the 3′ sequence of the probe determined to identify a particular typable locus. An ASPE reaction can include 1, 2, or 3 different MM probes, for example, at discrete array locations, the number being chosen depending upon the diversity occurring at the particular locus being assayed. For example, two probes can be used to determine which of 2 alleles for a particular locus are present in a sample, whereas three different probes can be used to distinguish the alleles of a 3-allele locus.

In particular embodiments, an ASPE reaction can include a nucleotide analog that is derivatized to be chain terminating. Thus, a PM probe in a probe-fragment hybrid can be modified to incorporate a single nucleotide analog without further extension. Exemplary chain terminating nucleotide analogs include, without limitation, those set forth above in regard to the SBE reaction. Furthermore, one or more nucleotides used in an ASPE reaction whether or not they are chain terminating can include a detection label such as those described previously herein. If desired, more than one nucleotide in an ASPE reaction can be labeled.

Pyrosequencing is an extension assay that can be used to add one or more nucleotides to a detection position(s); it is similar to SBE except that identification of typable loci is based on detection of a reaction product, pyrophosphate (PPi), produced during the addition of a dNTP to an extended probe, rather than on a label attached to the nucleotide. One molecule of PPi is produced per dNTP added to the extension primer. That is, by running sequential reactions with each of the nucleotides, and monitoring the reaction products, the identity of the added base is determined. Pyrosequencing can be used in the invention using conditions such as those described in U.S. 2002/0001801, hereby expressly incorporated by reference.

In some embodiments, detection of typable loci can include amplification of target oligonucleotides following formation of probe-target hybrids, resulting in a significant increase in the number of target molecules. Target amplification-based detection techniques can include, for example, the polymerase chain reaction (PCR), strand displacement amplification (SDA), or nucleic acid sequence based amplification (NASBA). Alternatively, rather than amplify the target, alternate techniques can use the target as a template to replicate a hybridized probe, allowing a small number of target molecules to result in a large number of signaling probes, that then can be detected. Probe amplification-based strategies include, for example, the ligase chain reaction (LCR), cycling probe technology (CPT), invasive cleavage techniques such as Invader™ technology, Q-Beta replicase (QβR) technology or sandwich assays. Such techniques can be carried out, for example, under conditions described in U.S. Ser. Nos. 60/161,148, 09/553,993 and 090/556,463; and U.S. Pat. No. 6,355,431 B1, all of which are hereby expressly incorporated by reference, or as set forth below. Such amplification techniques can include a step of attaching the target oligonucleotide or amplification primers to a solid phase substrate. Attachment to a solid phase substrate can be convenient for washing away impurities, if desired, prior to detection on an array of the invention.

Detection with oligonucleotide ligation amplification (OLA) involves the template-dependent ligation of two smaller probes into a single long probe, using a target sequence as the template. In a particular embodiment, a single-stranded target sequence includes a first target domain and a second target domain, which are adjacent and contiguous. A first OLA probe and a second OLA probe can be hybridized to complementary sequences of the respective target domains. The two OLA probes are then covalently attached to each other to form a modified probe. In embodiments where the probes hybridize directly adjacent to each other, covalent linkage can occur via a ligase. In one embodiment one of the ligation probes may be attached to a surface such as an array or a particle. In another embodiment both ligation probes may be attached to a surface such as an array or a particle.

Alternatively, an extension ligation (GoldenGate™) assay can be used wherein hybridized probes are non-contiguous and one or more nucleotides are added along with one or more agents that join the probes via the added nucleotides. Exemplary agents include, for example, polymerases and ligases. If desired, hybrids between modified probes and targets can be denatured, and the process repeated for amplification leading to generation of a pool of ligated probes. As above, these extension-ligation probes can be, but need not be, attached to a surface such as an array or a particle. Further conditions for extension ligation assay that are useful in the invention are described, for example, in U.S. Pat. No. 6,355,431 B1 and U.S. application Ser. No. 10/177,727, hereby expressly incorporated by reference.

OLA is referred to as the ligation chain reaction (LCR) when double-stranded target oligonucleotides are used. In LCR, the target sequence can be denatured, and two sets of probes added: one set as outlined above for one strand of the target, and a separate set (i.e. third and fourth primer probe nucleic acids) for the other strand of the target. Conditions can be used in which the first and second probes hybridize to the target and are modified to form an extended probe. Following denaturation of the target-modified probe hybrid, the modified probe can be used as a template, in addition to the second target sequence, for the attachment of the third and fourth probes. Similarly, the ligated third and fourth probes can serve as a template for the attachment of the first and second probes, in addition to the first target strand. In this way, an exponential, rather than just a linear, amplification can occur when the process of denaturation and ligation is repeated.

The modified OLA probe product can be detected in any of a variety of ways. In a particular embodiment, a template-directed probe modification reaction can be carried out in solution and the modified probe hybridized to a locus-specific capture probe in an array. A capture probe is generally complementary to at least a portion of the modified OLA probe. In an exemplary embodiment, the first OLA probe can include a detectable label and the second OLA probe can be substantially complementary to the capture probe. A non-limiting advantage of this embodiment is that artifacts due to the presence of labeled probes that are not modified in the assay are minimized because the unmodified probes do not include the complementary sequence that is hybridized by the capture probe. An OLA detection technique can also include a step of removing unmodified labeled probes from a reaction mixture prior to contacting the reaction mixture with a capture probe as described, for example, in U.S. Pat. No. 6,355,431 B1, hereby expressly incorporated by reference.

Alternatively, a genome fragment target can be immobilized on a solid-phase surface and a reaction to modify hybridized OLA probes performed on the solid phase surface. Unmodified probes can be removed by washing under appropriate stringency. The modified probes can then be eluted from the genome fragment target using denaturing conditions, such as, 0.1 N NaOH, and detected as described herein. Other conditions in which target oligonulceotides can be detected when used in an OLA technique include, for example, those described in U.S. Pat. Nos. 6,355,431 B1, 5,185,243, 5,679,524 and 5,573,907; EP 0 320 308 B1; EP 0 336 731 B1; EP 0 439 182 B1; WO 90/01069; WO 89/12696; WO 97/31256; and WO 89/09835, and U.S. Ser. Nos. 60/078,102 and 60/073,011, all of which are hereby expressly incorporated by reference.

Target oligonucleotides can be detected in a method of the invention using rolling circle amplification (RCA). In a first embodiment, a single probe can be hybridized to a target oligonucleotide such that the probe is circularized while hybridized to the target. Each terminus of the probe hybridizes adjacently on the target and addition of a polymerase results in extension of the circular probe. However, since the probe has no terminus, the polymerase continues to extend the probe repeatedly. This results in amplification of the circular probe. Following RCA the amplified circular probe can be detected. This can be accomplished in a variety of ways; for example, the primer can be labeled or the polymerase can incorporate labeled nucleotides and labeled product detected by a locus-specific capture probe in a detection array. Rolling-circle amplification can be carried out under conditions such as those generally described in Baner et al. (1998) Nuc. Acids Res. 26:5073-5078; Barany, F. (1991) Proc. Natl. Acad. Sci. USA 88:189-193; and Lizardi et al. (1998) Nat Genet. 19:225-232, all hereby expressly incorporated by reference.

Furthermore, rolling circle probes used in the invention can have structural features that render them unable to be replicated when not annealed to a target. For example, one or both of the termini that anneal to the target can have a sequence that forms an intramolecular stem structure, such as a hairpin structure. The stem structure can be made of a sequence that allows the open circle probe to be circularized when hybridized to a legitimate target sequence but results in inactivation of uncircularized open circle probes. This inactivation reduces or eliminates the ability of the open circle probe to prime synthesis of a modified probe in a detection assay or to serve as a template for rolling circle amplification. Exemplary probes capable of forming intramolecular stem structures and methods for their use which can be used in the invention are described in U.S. Pat. No. 6,573,051, hereby expressly incorporated by reference.

In another embodiment, detection can include OLA followed by RCA. In this embodiment, an immobilized primer can be contacted with a target oligonucleotide. Complementary sequences will hybridize with each other resulting in an immobilized duplex. A second primer can also be contacted with the target oligonucleotide. The second primer hybridizes to the target oligonucleotide adjacent to the first primer. An OLA reaction can be carried out to attach the first and second primer as a modified primer product, for example, as described above. The target oligonucleotide can then be removed and the immobilized modified primer product hybridized with an RCA probe that is complementary to the modified primer product but not the unmodified immobilized primer. An RCA reaction can then be performed.

In a particular embodiment, a padlock probe can be used both for OLA and as the circular template for RCA. Each terminus of the padlock probe can contain a sequence complementary to a target oligonucleotide. More specifically, the first end of the padlock probe can be substantially complementary to a first target domain, and the second end of the RCA probe can be substantially complementary to a second target domain, adjacent to the first domain. Hybridization of the padlock probe to the target oligonucleotide results in the formation of a hybridization complex. Ligation of the discrete ends of a single oligonucleotide results in the formation of a modified hybridization complex containing a circular probe that acts as an RCA template complex. Addition of a polymerase to the RCA template complex can allow formation of an amplified product nucleic acid. Following RCA, the amplified product nucleic acid can be detected, for example, by hybridization to an array either directly or indirectly and an associated label detected.

A padlock probe used in the invention can further include other characteristics such as an adapter sequence, restriction site for cleaving concatemers, a label sequence, or a priming site for priming the RCA reaction as described, for example, in U.S. Pat. No. 6,355,431 B1. This same patent also describes padlock probe methods that can be used to detect target sequences in a method of the invention.

A variation of LCR that can be used to detect a target sequence in a method of the invention utilizes chemical ligation under conditions such as those described in U.S. Pat. Nos. 5,616,464 and 5,767,259, both of which are expressly incorporated by reference. In this embodiment, similar to enzymatic modification, a pair of probes can be utilized, wherein the first probe is substantially complementary to a first domain of a target oligonucleotide and the second probe is substantially complementary to an adjacent second domain of the target. Each probe can include a portion that acts as a “side chain” that forms one half of a non-covalent stem structure between the probes rather than binding the target sequence. Particular embodiments utilize substantially complementary nucleic acids as the side chains. Thus, upon hybridization of the probes to the target sequence, the side chains of the probes are brought into spatial proximity. At least one of the side chains can include an activatable cross-linking agent, generally covalently attached to the side chain, that upon activation, results in a chemical cross-link or chemical ligation with the adjacent probe. The activatible group can include any moiety that will allow cross-linking of the side chains, and include groups activated chemically, photonically or thermally, such as photoactivatable groups. In some embodiments a single activatable group on one of the side chains is enough to result in cross-linking via interaction to a functional group on the other side chain; in alternate embodiments, activatable groups can be included on each side chain. One or both of the probes can be labeled.

Once a hybridization complex is formed, and the cross-linking agent has been activated such that the probes have been covalently attached to each other, the reaction can be subjected to conditions to allow for the disassocation of the hybridization complex, thus freeing up the target to serve as a template for the next ligation or cross-linking. In this way, signal amplification can occur, and the cross-linked products can be detected, for example, by hybridization to an array either directly or indirectly and an associated label detected.

In particular embodiments, amplification-based detection can be achieved using invasive cleavage technology. Using such an approach, a genome fragment target can be hybridized to two distinct probes. The two probes are an invader probe, which is substantially complementary to a first portion of the genome fragment target, and a signal probe, which has a 3′ end substantially complementary to a sequence having a detection position and a 5′ non-complementary end which can form a single-stranded tail. The tail can include a detection sequence and typically also contains at least one detectable label. However, since a detection sequence in a signal probe can function as a target sequence for a locus-specific capture probe, sandwich configurations utilizing label probes can be used as described herein and the signal probe need not include a detectable label.

Hybridization of the invader and signal probes near or adjacent to one another on a target oligonucleotide can form any of several structures useful for detection of the probe-fragment hybrid. For example, a forked cleavage structure can form, thereby providing a substrate for a nuclease which cleaves the detection sequence from the signal probe. The site of cleavage is controlled by the distance or overlap between the 3′ end of the invader probe and the downstream fork of the signal probe. Therefore, neither oligonucleotide is cleaved when misaligned or when unattached to a genome fragment target. Exemplary nucleases that can be used include, without limitation, those derived from Thermus aquaticus, Thermus flavus, or Thermus thernophilus; those described in U.S. Pat. Nos. 5,719,028 and 5,843,669, hereby expressly incorporated by reference, or Flap endonucleases (FENs) as described, for example, in U.S. Pat. No. 5,843,669 and Lyamichev et al., Nature Biotechnology 17:292-297 (1999), hereby expressly incorporated by reference. If desired, the 3′ portion of a cleaved signal probe can be extracted, for example, by binding to a solid-phase capture tag such as bead bound streptavidin, or by crosslinking through a capture tag to produce aggregates. The 5′ detection sequence of a signal probe, can be detected using methods set forth below such as hybridization to a probe on an array. Invasive cleavage technology can further be used in the invention using conditions and detection methods described, for example, in U.S. Pat. Nos. 6,355,431; 5,846,717; 5,614,402; 5,719,028; 5,541,311; or 5,843,669, hereby expressly incorporated by reference.

A further amplification-based detection technique that can be used to detect target sequences is cycling probe technology (CPT). A CPT probe can include two probe sequences separated by a scissile linkage. The CPT probe is substantially complementary to a genome fragment target sequence and thus will hybridize to it to form a probe-fragment hybrid. The CPT probe can be hybridized to a genome fragment target in a method of the invention. Typically the temperature and probe sequence are selected such that the primary probe will bind and shorter cleaved portions of the primary probe will dissociate. Depending upon the particular application, CPT can be done in solution, or either the target or scissile probe can be attached to a solid support. A probe-fragment hybrid formed in the methods can be subjected to cleavage conditions which cause the scissile linkage to be selectively cleaved, without cleaving the target sequence, thereby separating the two probe sequences. The two probe sequences can then be disassociated from the target. In particular embodiments, excess probe can be used and the reaction allowed to be repeated any number of times such that the effective amount of cleaved probe is amplified.

Any linkage within a CPT probe that can be selectively cleaved when the probe is part of a hybridization complex, that is, when a double-stranded complex is formed can be used as a scissile linkage. Any of a variety of scissile linkages can be used in the invention including, for example, RNA which can be cleaved when in a DNA:RNA hybrid by various double-stranded nucleases such as ribonucleases. Such nucleases will selectively nick or excise RNA nucleosides from a RNA:DNA hybridization complex rather than DNA in such a hybrid or single stranded DNA. Further examples of scissile linkages and cleaving agents that can be used in the invention are described in U.S. Pat. No. 6,355,431 B1 and references cited therein.

Cleaved probes produced by a CPT reaction can be detected using methods such as hybridization to an array or other methods set forth herein. For example, a cleaved probe can be bound to a locus-specific capture probe, either directly or indirectly, and an associated label detected. CPT technology can be carried out under conditions described, for example, in U.S. Pat. Nos. 5,011,769; 5,403,711; 5,660,988; and 4,876,187, and PCT published applications WO 95/05480; WO 95/1416, and WO 95/00667, and U.S. Ser. No. 09/014,304, all hereby expressly incorporated by reference.

In particular embodiments, CPT with a probe containing a scissile linkage can be used to detect mismatches, as is generally described in U.S. Pat. No. 5,660,988, and WO 95/14106, hereby expressly incorporated by reference. In such embodiments, the sequence of the scissile linkage can be placed at a position within a longer sequence that corresponds to a particular sequence to be detected, i.e. the area of a putative mismatch. In some embodiments of mismatch detection, the rate of generation of released fragments is such that the methods provide, essentially, a yes/no result, whereby the detection of virtually any released fragment indicates the presence of a desired typable locus. Alternatively or additionally, the final amount of cleaved fragments can be quantified to indicate the presence or absence of a target sequence.

Target sequences can also be detected in a method of the invention using a sandwich assay. A sandwich assay is an amplification-based technique in which multiple probes, typically labeled, are bound to a single target oligonucleotide. In an exemplary embodiment a target oligonucleotide can be bound to a solid substrate via a complementary locus-specific capture probe. Typically, a unique capture probe will be present for each typable locus sequence to be detected. In the case of a bead array, each bead can have one of the unique locus-specific capture probes. If desired, capture extender probes can be used, that allow a universal surface to have a single type of capture probe that can be used to detect multiple target sequences. Capture extender probes include a first portion that will hybridize to all or part of the capture probe, and a second portion that will hybridize to a first portion of the target sequence to be detected. Accordingly customized soluble probes can be generated, which as will be appreciated by those in the art can simplify and reduce costs in many applications of the invention. In particular embodiments, two capture extender probes can be used. This can provide a non-limiting advantage of stabilizing assay complexes, for example, when a target sequence to be detected is large, or when large amplifier probes (particularly branched or dendrimer amplifier probes) are used.

Once a target oligonucleotide has been bound to a solid substrate, such as a bead, via a locus-specific capture probe, an amplifier probe can be hybridized to the target oligonucleotide to form a probe-target hybrid. Exemplary amplifier probes that can be used in a method of the invention and conditions for their use in sandwich assays are described in U.S. Pat. No. 6,355,431. Briefly, an amplifier probe is a nucleic acid having at least one probe sequence, and at least one amplification sequence. A first probe sequence of an amplifier probe can be used, either directly or indirectly, to hybridize to a genome fragment target sequence. An amplification sequence of an amplifier probe can be any of a variety of sequences that are used, either directly or indirectly, to bind to a first portion of a label probe. Typically an amplifier probe will include a plurality of amplification sequences. The amplification sequences can be linked to each other in a variety of ways including, for example, covalently linked directly to each other, or to intervening sequences or chemical moieties.

Label probes comprising detectable labels can hybridize to target oligonucleotide thereby forming probe-target hybrids and the labels can be detected to determine the presence of typable loci. The amplification sequences of the amplifier probe can be used, either directly or indirectly, to bind to a label probe to allow detection. Detection of the amplification reactions of the invention, including the direct detection of amplification products and indirect detection utilizing label probes (i.e. sandwich assays), can be done by detecting assay complexes having labels. Exemplary methods for using a sandwich assay and associated nucleic acids that can be used in the present invention are further described in U.S. Ser. No. 60/073,011 and in U.S. Pat. Nos. 6,355,431; 5,681,702; 5,597,909; 5,545,730; 5,594,117; 5,591,584; 5,571,670; 5,580,731; 5,571,670; 5,591,584; 5,624,802; 5,635,352; 5,594,118; 5,359,100; 5,124,246 and 5,681,697, all of which are hereby expressly incorporated by reference.

Depending upon a particular application of the methods of the invention, the detection techniques set forth above can be used to detect primary genome fragment targets or to detect targets in an amplified representative population of genome fragments.

In particular embodiments, it can be desirable to remove unextended or unreacted nucleic acids from a reaction mixture prior to detection since unextended or unreacted primers can often compete with the modified probes during detection, thereby diminishing the signal. The concentration of the unmodified probes relative to modified probes can often be relatively high, for example, in embodiments where a large excess of probe is used. Accordingly, a number of different techniques can be used to facilitate the removal of unextended primers. Exemplary methods that can be used to remove unextended primers include, for example, those described in U.S. Pat. No. 6,355,431.

The invention includes methods for making first and second locus-specific arrays, wherein two different chimeric oligonucleotides, each having a different locus-specific portion, are contacted with the adapter probes of duplicate universal array. Such arrays are made by hybridizing different chimeric oligonucleotides to the adapter probes of a universal array. This can be done, for example, by using a universal array having two different adapter probes. A population of chimeric oligonucleotides having an adapter-specific sequence that is complementary to the first adapter probe, and a second population having an adapter-specific sequence that is complementary to the second adapter probe can both be hybridized to the universal array and the universal array converted to a locus-specific array by one of the methods of the invention.

The invention also includes locus-specific arrays comprising an adapter probe covalently attached to a solid support, and a chimeric oligonucleotide comprising an adapter-specific portion and a locus-specific portion. The adapter-specific portion of the chimeric oligonucleotide is hybridized to the adapter probe which is attached to the solid support, thus making the locus-specific portion available for hybridization to target.

The invention includes locus-specific arrays comprising an adapter probe covalently attached to a solid support, a locus-specific oligonucleotide, and a locus splint oligonucleotide comprising an adapter-specific portion and a locus-specific portion. The adapter-specific portion of the splint oligonucleotide is hybridized to the adapter probe which is attached to the solid support, and the locus-specific portion is hybridized to the locus-specific oligonucleotide which can be a third oligonucleotide containing a locus specific sequence or a chimeric oligonucleotide. The locus-specific oligonucleotide and the adapter probe can further be ligated together to form an extended adapter probe containing a locus-specific region.

The invention includes locus-specific arrays comprising an adapter probe covalently attached to a solid support, a locus-specific oligonucleotide, and a splint oligonucleotide comprising a portion specific for a first adapter sequence and a portion specific for a second adapter sequence. The first adapter-specific portion of the splint oligonucleotide is hybridized to the adapter probe which is attached to the solid support, and the second adapter specific portion is hybridized to an adapter portion of a chimeric oligonucleotide containing a locus-specific portion and an adapter portion. The locus-specific oligonucleotide and the adapter probe can further be ligated together to form an extended adapter probe containing a locus-specific region.

The invention includes locus-specific arrays comprising an adapter probe covalently attached to a solid support, a chimeric oligonucleotide comprising an adapter-specific portion and a locus-specific portion. The adapter-specific portion of the chimeric oligonucleotide is hybridized to the adapter probe which is attached to the solid support, thus making the locus-specific portion available for hybridization to a locus-specific oligonucleotide.

The invention also includes locus-specific arrays comprising an adapter probe covalently attached to a solid support and a chimeric oligonucleotide comprising an adapter-specific adapter-probe portion and a locus-specific portion. The adapter-specific-probe portion of the chimeric oligonucleotide is crosslinked to the adapter probe, resulting in an extended adapter probe having a locus-specific portion available for hybridization to a locus-containing oligonucleotide.

The invention includes methods for using the locus-specific arrays of the invention for detecting target nucleic acid sequences in a test sample. Target sequences can be detected using methods described herein, and methods known to those of skill in the art.

The present invention contemplates diagnostic systems for carrying out one or more of the methods described previously herein. A diagnostic system of the invention can be provided in kit form including, if desired, a suitable packaging material. In one embodiment, for example, a diagnostic system can include a plurality of adapter probes, for example, in an array format, and one or more reagents useful for detecting a target nucleic acid hybridized to a probe of the array. Accordingly, any combination of reagents or components that is useful in a method of the invention, such as those set forth herein previously in regard to particular methods, can be included in a kit provided by the invention. For example, a kit can include one or more universal arrays containing adapter probes bound to an array, chimeric oligonucleotides having a region complementary to the probes and a locus specific portion, splint oligonucleotides, oligonucleotides having sequences complementary to splint oligonucleotides, a ligase, a polymerase, buffers or a subcombination of these components.

As used herein, the phrase “packaging material” refers to one or more physical structures used to house the contents of the kit, such as nucleic acid probes, oligonucleotides or the like. The packaging material can be constructed by well-known methods, preferably to provide a sterile, contaminant-free environment. The packaging materials employed herein can include, for example, those customarily utilized in nucleic acid-based diagnostic systems. Exemplary packaging materials include, without limitation, glass, plastic, paper, foil, and the like, capable of holding within fixed limits a component useful in the methods of the invention such as an isolated nucleic acid or other oligonucleotide.

The packaging material can include a label which indicates that the invention nucleic acids can be used for a particular method. For example, a label can indicate that the kit is useful for detecting a particular set of target nucleic acids.

Instructions for use of the packaged reagents or components are also typically included in a kit of the invention. “Instructions for use” typically include a tangible expression describing the reagent or component concentration or at least one assay method parameter, such as the relative amounts of kit components and sample to be admixed, maintenance time periods for reagent/sample admixtures, temperature, buffer conditions, and the like.

### EXAMPLES

The present invention is further illustrated by the following examples, which should not be construed as limiting in any way.

### Example 1

### “Sandwich” Hybridization Extension of Adapter Probe

Adapter-probes on a universal array are converted into locus-specific oligonucleotides by hybridization to chimeric oligonucleotides that contain both an adapter-specific sequence and a locus-specific sequence. The resulting hybrid is tested by evaluating the ability of the extended adapter probe to detect labeled target oligonucleotides.

Chimeric oligonucleotides containing an adapter-specific portion and a locus-specific portion are hybridized to a universal array having DNA adapter probe attached at assay locations. The chimeric oligonucleotide is synthesized with a psoralen moiety at its 5′ end. Psoralen can also be incorporated internally or at the 3′ end of an oligonucleotide by modifying an amine-labeled oligo with psoralen-NHS (Pierce). Typically, the psoralen is placed adjacent to an AT dinucleotide on the adapter-specific sequence. Chimeric oligonucleotides are hybridized at high stringency (40% formamide, room temperature) at 6-50 nM for 1 hour, under saturating conditions, to the adapter-probe arrays.

After washing the hybridization complex, cross-linking is carried out by exposing the duplex-probe array (1×SSPE buffer present, array sitting on ice) to long wavelength UV light, wherein the intercalated psoralen moiety cross-links the two thymidine bases located on opposing DNA strands (Bornet et al. 1995). The UV exposure used is 6 J/cm2 of 365 nm (long UV) applied to the outer glass surface of the DNA array.

The efficiency of hybridization is evaluated by hybridizing Cy3 fluorescently-labeled target oligonucleotides, having sequences complementary to the locus-specific portion of the adapter probe, to the chimeric oligonucleotide of the hybridization complex. The gene extension products on the beads are incubated in 6× hybridization buffer (1000 mM NaCl, 100 mM potassium phosphate, 0.1% Tween-20, pH 7.6) for 1 minute, then incubated with 6 nM Cy3-labeled target oligonucleotides in 6× hybridization buffer having 40% formamide at RT for 30 mins. The beads are then washed with 20% formamide, 2 times with 6× hybridization buffer, and imaged to determine the signal intensities.

The results obtained using the target oligonucleotides are then compared, to estimate the frequency of hybridization relative to the number of hybridizable sites (adapter probes) originally available. Next, the data is compared to data obtained using the standard Z50 array. The Z50 array technology is described in Dickinson et al., Genetic Engineering News 23: 20 (2003), hereby expressly incorporated by reference. The data show that target detection using the ligation-extended crosslinked locus-specific array is comparable to that observed using a standard Z50 array.

### Example II

### Polymerase Extension of Adapter Probe

A universal DNA array was converted into a locus-specific array by polymerase extension of the adapter probe. The efficiency of polymerase extension was tested by evaluating the ability of the extended adapter probe to detect labeled target oligonucleotides.

A pool of ninety-six 76-mer chimeric oligonucleotides, each containing a different adapter-specific portion and a different locus-specific portion, was hybridized to a universal array. The universal array contained bead types having the adapter probe oligonucleotides represented in the chimeric oligonucleotide pool. The orientation of probes on the universal array was such that these probes could be used as primers in a polymerase extension reaction given a suitably hybridized chimeric oligonucleotide.

Chimeric oligonucleotides were also gel-purified and tested in the extension assay, to examine the effect of removing truncated oligonucleotides from the annealing mix. The gel-purified chimeric oligonucleotides were purified “en-masse” in a single polyacrylamide gel well. Chimeric oligonucleotides, either unpurified or gel-purified, were hybridized at high stringency (40% formamide, room temperature) at 6 nM for 1 hour, under saturating conditions, to the adapter probe bead arrays. The beads were hybridized to gel-purified gene extension oligos (6 nM) in 6× hybridization buffer with 40% formamide. The chimeric oligonucleotides were heat-denatured at 95° C. for 10 minutes and hybridized at RT for 1 hour.

After hybridization, the arrays were washed with 6× hybridization buffer at room temperature for 1 minute, in 1× hybridization buffer at room temperature for 1 minute, in 100% isopropanol for 30 seconds, then incubated at 37° C. for 20 minutes.

Primer extension was carried out using Klenow exo(−) and standard dNTPs. Pre-extension buffer was prepared by combining, per 20 μl reaction volume, 10×T4 DNA polymerase buffer (add 2 μl, to a final concentration of 1×); water (16 μl); 10% Tween-20 (1 μl, to a final concentration of 0.1%); and 10 mg/ml BSA (1 μl, to a final concentration of 100 μg/ml).

Oligonucleotide extension solutions were prepared by combining, per 20 μl reaction volume, 10×T4 DNA polymerase buffer (2 μl, to a final concentration of 1×); 25 mM MgCl (4 μl, to a final concentration of 5 Mm); 1 mM dNTPs (2 μl to a final concentration of 100 μM); 10 mg/ml BSA (1 μl, to a final concentration of 100 g/ml); 10% Tween-20 (1 μl, to a final concentration of 0.1%); 100 mM DTT (0.2 μl, to a final concentration of 1 mM); 5 U/μl Klenow enzyme (0.2 μl, a total of 1 U); and water (11.2 μl).

The arrays were dipped in 37° C. equilibrated 1× pre-extension buffer for 1 minute, in 37° C. equilibrated 1× extension buffer for 15 minutes, in 0.1N NaOH (fresh) for 1 minute, in 6× hybridization buffer for 1 minute, then again in 6× hybridization buffer for 1 minute.

Extension was evaluated by hybridizing mock target oligonucleotides consisting of either Set A, Set B or Set C fluorescently-labeled target oligonucleotides. Set A oligonucleotides were complementary to the adapter probe. Set B oligonucleotides were complementary to the distal end of the fully-extended adapter probe. Set C oligonucleotides were complementary to the entire polymerase-extended (i.e., locus-specific) portion of the fully-extended adapter probe.

Set A and B target oligonucleotides were used with two bead types, 208 and 210, to evaluate the extension efficiency of the reactions. Set A oligonucleotide signal intensities represented the amount of hybridization substrate (adapter oligonucleotide) originally available in the extension reactions. Set B oligonucleotide signal intensities represented the amount of extended product generated. Comparing Set A and Set B signal intensities indicated the proportion of available substrate that became fully extended.

The detection reactions were performed by dipping the beads in 6× hybridization buffer for 1 minute, then hybridizing them with Cy3-labeled 10 nM Set A or B oligonucleotides in 6× hybridization buffer with 40% formamide at room temperature for 30 minutes. The beads were then washed once with 20% formamide and twice with 6× hybridization buffer. The signal intensities were then determined.

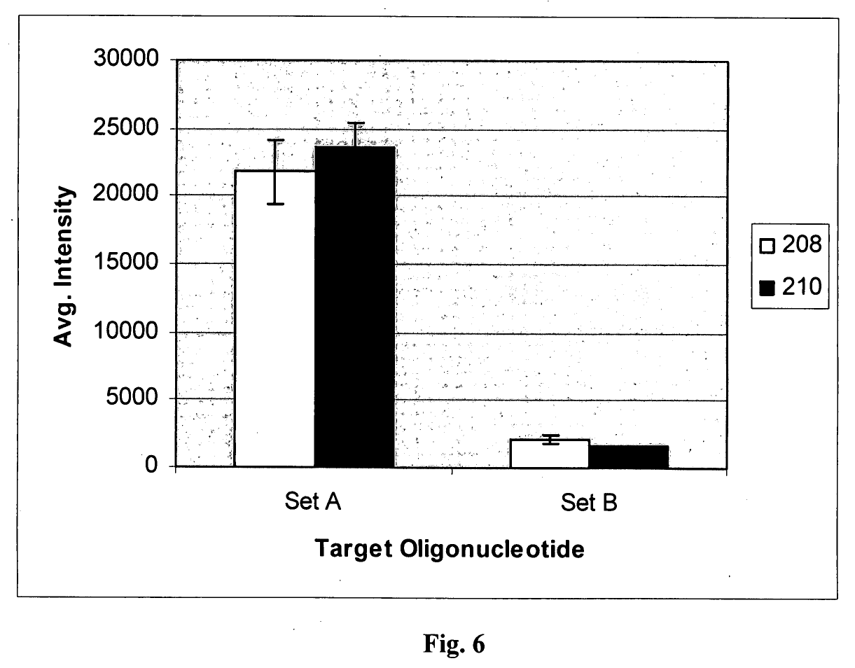

The results obtained using the Set A and Set B target oligonucleotides to estimate the frequency of polymerase extension relative to the number of hybridizable sites available are shown in FIG. 6. The white bars show data for bead type 208, and the black bars for bead type 210. The first two bars on the left show the amount of hybridization of Set A target oligonucleotides Dc_CG0971_{—}1 (5′-GATAATGATTATCATCTACATATCACAACG-3′) and Dc_CG0971_{—}10 (5′-TTTGTCGCTCCATGCGCTTG-3′) to the adapter probe sequences on bead types 208 and 210, respectively. The two bars on the right of the graph show the amount of hybridization of Set B target oligonucleotides Dc_CG0971_{—}1_{—}11123304 (5′-TAGTGCCGGTATGATCGCTAACC-3′) and Dc_CG0971_{—}1_{—}11123321 (5′-TTCGCACTACCGAGCCGAGTTGT-3′) to the adapter probe sequences on bead types 208 and 210, respectively.

As shown, about 10% of the hybridizable sites were extended to full-length gene expression probes. The polymerase extension efficiency was perhaps influenced by the ultra-high density of capture probes on the arrays.

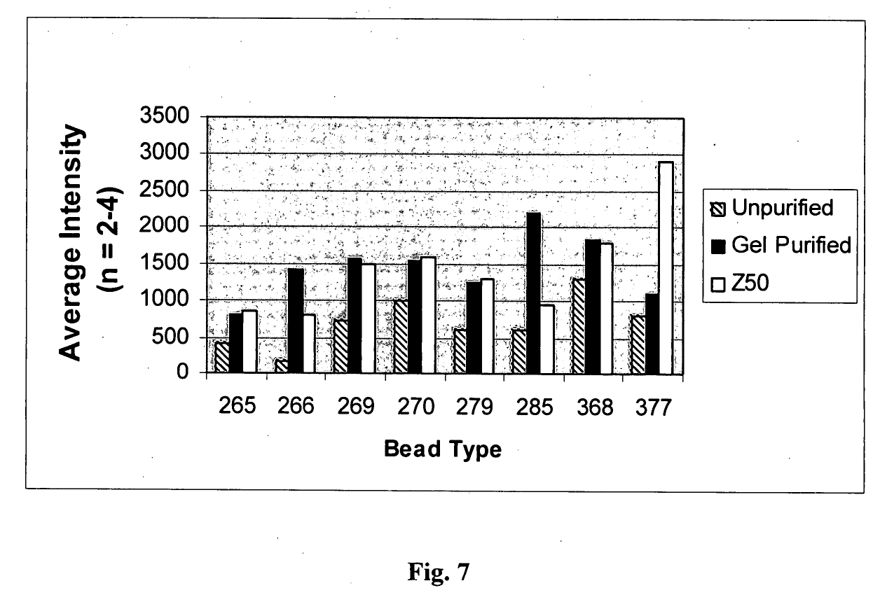

Next, the gene expression data were compared with data obtained using the standard Z50 array. Set C detector oligos (a mix of 12 oligos at 1 nM, 100 pM, and 10 pM) were hybridized for 1 hour in hybridization buffer/40% formamide at room temperature. The comparison of the resulting analytical intensities is shown in FIG. 7. Thus, the in-situ arrays made using gel-purified chimeric oligonucleotides performed similarly to the current Z50 gene expression arrays.

### Example III

### Extension of Adapter Probe Using Enzyme Ligation

Adapter probe oligonucleotides of a universal array are converted into locus-specific oligonucleotides by hybridizing a third oligonucleotide to a hybridization complex generated as in Example I. The third oligonucleotide sequence is complementary to a locus-specific sequence of the chimeric oligonucleotide. Hybridization is carried out under stringent conditions, and the resulting hybrid is washed.

The 5′ end of the third oligonucleotide is ligated to the 3′ end of the adapter probe oligonucleotide using T4 DNA ligase. The T4 DNA ligation buffer consists of the following: 50 mM Tris-HCl (pH 7.8), 10 mM MgCl2, 10 mM DTT, 1 mM ATP, 50 μg/ml BSA, 100 mM NaCl, 0.1% TX-100 and 2.0 U/μl T4 DNA ligase (New England Biolabs). Reactions are performed at 30° C. The ligation reactions are incubated from 2 to 16 hours.

Following ligation, arrays are washed 5-10 times with 1×SSPE (pH 7.4, 22° C.) on a GeneChip fluidics station, stained for 5 min with streptavidin-phycoerythrin conjugate (Molecular Probes, 2 ng/μl in 12 SSPE, 50 μg/ml BSA) on a rotating rotisserie at 22° C. and washed another 5-10 times with 1×SSPE.

The chimeric oligonucleotide is then denatured from the hybrid in 0.1 NaOH or 95% form amide at room temperature and removed from the array. The efficiency of ligation extension is evaluated by hybridizing fluorescently-labeled target oligonucleotides, having sequences that are complementary to the locus-specific portion of the adapter probe, to the extended-adapter probe, as described in Example III.

The results obtained using the target oligonucleotides are compared, to estimate the efficiency of ligation extension relative to the number of hybridizable sites (adapter probes) originally available. Next, the data is compared to data obtained using the standard Z50 array. These data show that target detection using the ligation-extended locus-specific array is comparable to that observed using a standard Z50 array.

Throughout this application various publications, patents and patent applications have been referenced. The disclosure of these publications, patents and patent applications in their entireties are hereby incorporated by reference in this application in order to more fully describe the state of the art to which this invention pertains.

Various embodiments of the invention have been described broadly and generically herein. Each of the narrower species and subgeneric groupings falling within the generic disclosure also form the part of these inventions. This includes within the generic description of each of the inventions a proviso or negative limitation that will allow removing any subject matter from the genus, regardless of whether or not the material to be removed was specifically recited.

Although the invention has been described with reference to the examples provided above, it should be understood that various modifications can be made without departing from the invention. Therefore, it is to be understood that within the scope of the appended claims, the invention may be practiced other than as specifically described.

1. A method of making a locus-specific array, comprising the following steps:
(a) providing a universal array having a plurality of assay locations wherein each of the assay locations comprises an adapter probe;
(b) providing a plurality of chimeric oligonucleotides;
(c) contacting the chimeric oligonucleotides with the adapter probes under conditions for forming a plurality of chimeric-oligonucleotide:adapter hybrids; and
(d) converting the hybrids into locus-specific assay locations of the locus-specific array.

2. The method of claim 1 wherein one or more of the chimeric oligonucleotides employed is at least 6 to 100 nucleic acid residues in length.

3. The method of claim 1, wherein each chimeric oligonucleotide comprises a locus-specific portion and an adapter-specific portion

4. The method of claim 3 wherein the locus-specific portion of one or more of the chimeric oligonucleotides is at least 6 to 100 nucleic acid residues in length.

5. The method of claim 3 wherein the adapter-specific portion of one or more of the chimeric oligonucleotides is at least 6 to 100 nucleic acid residues in length.

6. The method of claim 1 wherein one or more of the adapter probes used is at least 6 to 100 nucleic acid residues in length.

7. The method according to claim 1 further comprising:
(e) separating the chimeric oligonucleotide from the locus-specific array.

8. The method according to claim 1, wherein the chimeric oligonucleotides comprise synthetic molecules.

9. The method according to claim 1, wherein step (b) further comprises synthesizing the chimeric oligonucleotides.

10. The method according to claim 3, wherein the locus-specific sequence of the chimeric oligonucleotide is closer to the 5′ end of the chimeric oligonucleotide relative to the adapter-specific sequence.

11. The method according to claim 1, wherein the chimeric oligonucleotides are purified prior to being contacted with the adapter probes of the universal array.

12. The method according to claim 1, wherein at least two of the adapter probes on the universal array have different sequences.

13. The method according to claim 1, wherein the adapter probes of the universal array are covalently attached to particles.

14. The method according to claim 1, wherein step (c) comprises polymerase extension of the adapter probe.

15. The method according to claim 3, wherein the locus-specific portion of at least one of the chimeric oligonucleotides is separated from the adapter-specific portion by an intervening sequence.

16. The method according to claim 14, wherein step (c) further comprises ligation of the adapter probe to a third oligonucleotide comprising a locus-specific portion.

17. The method according to claim 1, wherein step (c) further comprises ligation of the adapter probe to a third oligonucleotide comprising a locus-specific portion.

18. The method according to claim 1, wherein the chimeric oligonucleotides comprise splint oligonucleotides, each comprising a first adapter-specific portion and portion specific for a locus specific oligonucleotide.

19. The method according to claim 18, wherein step (c) further comprises hybridization of at least one of the splint oligonucleotides to a chimeric oligonucleotide comprising a second adapter-specific portion and a locus-specific portion.

20. The method according to claim 19, wherein step (c) comprises ligation of the adapter probe to the third oligonucleotide.

21. The method according to claim 19, wherein step (c) further comprises polymerase extension of the adapter probe, thereby forming an extended adapter probe.

22. The method according to claim 20, wherein step (c) further comprises ligation of the extended adapter probe to the third oligonucleotide.

23. The method of claim 18, wherein step (c) comprises the sequential steps of
(i) hybridizing the locus-specific portion of at least one of the splint oligonucleotides to the locus-specific oligonucleotide; and
(ii) hybridizing the adapter-specific portion of at least one of the splint oligonucleotides to the adapter probes.

24. The method according to claim 18, wherein at least one of the splint oligonucleotides is hybridized in solution to a third oligonucleotide prior to contacting step (c).

25. The method according to claim 18 wherein step (c) comprises contacting the splint oligonucleotides with the adapter probe and locus-specific oligonucleotides under conditions for forming a plurality of ternary hybrids each comprising a adapter probe, splint oligonucleotide and third oligonucleotide.

26. The method according to claim 19, wherein step (d) comprises crosslinking the splint oligonucleotides to the third oligonucleotide.

27. The method according to claim 26, wherein psoralen is used as an agent to cross-link the splint oligonucleotide to the third oligonucleotide.

28. The method according to claim 1, wherein step (d) comprises crosslinking the chimeric oligonucleotide to the the adapter probe.

29. The method according to claim 28, wherein psoralen is used as an agent to cross-link the chimeric oligonucleotide to the adapter probe.

30. The method according to claim 16, wherein the ligation comprises enzymatic ligation.

31. The method according to claim 1, wherein the 3′ terminus of the adapter probe is covalently attached to its assay location.

32. A method of detecting a plurality of loci, comprising:
(a) providing a universal array having a plurality of assay locations wherein each of the assay locations comprises an adapter probe;
(b) providing a plurality of chimeric oligonucleotides, each chimeric oligonucleotide comprising a locus-specific portion and an adapter-specific portion;
(c) contacting the chimeric oligonucleotides with the adapter probes under conditions for forming a plurality of chimeric oligonucleotide:adapter probe hybrids;
(d) converting the hybrids into locus-specific assay locations of a locus-specific array; and
(e) contacting the locus-specific array with a plurality of target oligonucleotides, under conditions wherein target oligonucleotides that are complementary to sequences of locus-specific assay locations hybridize to the locus-specific assay locations, thereby detecting the target oligonucleotides.

33. A method of making a first and second locus-specific array, comprising the following steps:
(a) providing a first universal array having a plurality of assay locations wherein each of the assay locations comprises an adapter probe;
(b) providing a first plurality of chimeric oligonucleotides, each chimeric oligonucleotide comprising a locus-specific portion and an adapter-specific portion;
(c) contacting the first plurality of chimeric oligonucleotides with the adapter probes under conditions for forming a plurality of chimeric oligonucleotide:adapter-probe hybrids;
(d) converting the hybrids into locus specific assay locations of a first locus-specific array;
(e) providing a second universal array having a plurality of assay locations comprising the adapter probes;
(f) providing a second plurality of chimeric oligonucleotides comprising the adapter-specific portions and locus-specific portions, wherein the locus-specific portions of the first plurality of chimeric oligonucleotides are different from the locus-specific portions in the second plurality of chimeric oligonucleotides;
(g) contacting the second plurality of chimeric oligonucleotides with the adapter probes of the second universal array under conditions for forming a second plurality of chimeric oligonucleotide:adapter probe hybrids; and
(h) converting the second plurality of hybrids into locus-specific assay locations of a second locus-specific array.

34. The method according to claim 33, further comprising:
(i) contacting the first locus-specific array with a first plurality of target oligonucleotides, under conditions wherein target oligonucleotides that are complementary to sequences of locus-specific assay locations hybridize to the locus-specific assay locations, thereby detecting the target oligonucleotides.

35. The method according to claim 34, further comprising:
(j) contacting the second locus-specific array with a second plurality of target oligonucleotides, under conditions wherein target oligonucleotides that are complementary to sequences of locus-specific assay locations hybridize to the locus-specific assay locations, thereby detecting the target oligonucleotides.

36. A locus-specific array, comprising:
(a) an adapter probe covalently attached to a solid support; and
(b) a chimeric oligonucleotide comprising an adapter-specific portion and a locus-specific portion;
wherein the adapter-specific portion of the chimeric oligonucleotide is hybridized to the adapter probe.

37. A locus-specific array, comprising:
(a) an adapter probe covalently attached to a solid support;
(b) a locus-specific oligonucleotide; and
(c) a locus splint oligonucleotide comprising an adapter-specific portion and a locus-specific portion;
wherein the adapter-specific portion of the locus splint oligonucleotide is hybridized to the adapter probe and the locus-specific portion of the splint oligonucleotide is hybridized to the locus-specific oligonucleotide.

38. The locus-specific array of claim 37, wherein the adapter probe is ligated to the locus-specific oligonucleotide.

39. A locus-specific array comprising:
(a) an adapter probe covalently attached to a solid support; and
(b) a chimeric oligonucleotide comprising an adapter-specific portion and a locus-specific portion;
wherein the adapter-specific portion of the chimeric oligonucleotide is hybridized to the adapter probe.

40. A locus-specific array comprising:
(a) an adapter probe covalently attached to a solid support; and
(b) a chimeric oligonucleotide comprising an adapter-specific portion and a locus-specific portion;
wherein the adapter-specific portion of the chimeric oligonucleotide is crosslinked to the adapter probe.

41. The locus specific array of claim 40, wherein psoralen is used as an agent to cross-link the chimeric oligonucleotide to the adapter probe.
